(19) United States
(12) Patent Application Publication
(10) Pub. No.: US 2006/0122148 A1
(43) Pub. Date: Jun. 8, 2006

(54) High affinity receptors for helicobacter pylori and use thereof

(76) Inventors: Susann Teneberg, Hindas (SE); Halina Miller-Podraza, Vastra Frolunda (SE); Jari Natunen, Vantaa (FI); Karl-Anders Karlsson, Goteborg (SE)

Correspondence Address: BIRCH STEWART KOLASCH & BIRCH, PO BOX 747, FALLS CHURCH, VA, 22040-0747, US

(21) Appl. No.: 10/533,877

(22) PCT Filed: Nov. 6, 2003
(86) PCT No.: PCT/FI03/00840

§ 371 Date: Nov. 23, 2005

(30) Foreign Application Priority Data
Nov. 6, 2002 (FI) 20021989

(51) Int. Cl. A61K 31/739; C08B 37/00

(52) U.S. Cl. 514/54; 536/53

(57) ABSTRACT

The present invention describes an oligosaccharide substance or receptor binding to Helicobacter pylori, and the use thereof in, e.g., pharmaceutical and nutritional compositions for the treatment of conditions due to the presence of Helicobacter pylori. The invention is also directed to the use of the receptor for diagnostics of Helicobacter pylori.

### FIELD OF THE INVENTION

The present invention describes novel high affinity receptors for Helicobacter pylori. The receptors comprise sialylated poly-N-acetyllactosamine structures. The present invention is directed to therapheutic and prophylactic uses of the substances against the major gastric pathogen Helicobacter pylori. Furthermore the present invention is directed to analytic and diagnostic uses of the structures. The present invention is further directed to the uses of the structures in functional foods preventing H. pylori infections.

### BACKGROUND OF THE INVENTION

Sialyllactose and sialic acid on glycoproteins or glycolipids have been recognized as receptors for Helicobacter pylori. The receptor structure has been considered to be NeuNAcα3Gal- or NeuNAcα3Galβ4Glc (sialyl-lactose) (U.S. Pat. Nos. 5,883,079, 5,753,630 and 5,514,660 and, Mysore et al. 1999) or NeuNAcα3Galβ4GlcNAc (Johansson and Miller-Podraza 1998, Miller-Podraza et al., 1997). More recently fucosylated variants such as sialyl-Lewis x, NeuNAcα3Galβ4(Fucα3)GlcNAc (sLex), and especially a difucosylated structure sialyl-dimeric Lewis x, NeuNAcα3Galβ4(Fucα3)GlcNAcβ3Galβ4(Fucα3)GlcNAc (sdiLex) (Mahdavi et al., 2002), have been considered as Helicobacter ligands. When the structures are expressed on glycolipids sdiLex is orders of magnitude better receptor for H. pylori than sLex (Mahdavi et al., 2002). The present invention is directed to other larger, non-fucosylated poly-N-acetyllactosamine structures. The fucosylated and sialylated polylactosamines are good receptors for H. pylori. However, relatively high affinity epitopes can be represented on non-fucosylated poly-N-acetyllactosamines. The benefit of these structures is that there is no need to add fucose to the epitopes by chemical synthesis with 5-10 extra steps. The addition of fucose would also be industrially difficult as enzymatic or fermentative synthesis of fucosylated glycans has not been developed to commercial level yet.

The present invention further shows that NeuNAcα3Galβ4GlcNAc-structures need to comprise structure -β4Glc(NAc) for effective H. pylori binding. In general, similar glycolipid epitopes with other linkages to NeuNAcα3Galβ3GlcNAc and NeuNAcα3Galβ3GalNAc were not binding structures which observation is in contrast to what has been suggested for the terminal NeuNAcα3Gal-epitope.

The heptasaccharide glycolipid NeuNAcα3Galβ4GlcNAcβ3Galβ4GlcNAcβ3Galβ4GlcβCer have been shown to bind H. pylori (Roche. et al. 2001, Johansson and Miller-Podraza 1998, Miller-Podraza et al. 1997). The present invention shows that the non-fucosylated structure is actually more effective receptor for H. pylori than shorter NeuNAcα3Galβ4Glc(NAc) or sLex epitopes giving possibility to design lower cost high affinity inhibitors or diagnostic reagents for H. pylori. The present invention allows recognition of the sialylated polylactosamines as high affinity receptors among the known sialic acid comprising receptors. Furthermore, the present invention is directed to longer chain polylactosamines represented by glycolipid NeuNAcα3Galβ4GlcNAcβ3Galβ4GlcNAcβ3Galβ4GlcNAcβ3Galβ4GlcβCer and to branched polylactosamine structures such as presented by glycolipid NeuNAcα3Galβ4GlcNAcβ3(NeuNAcα3Galβ4GlcNAcβ6)Galβ4GlcNAcβ3Galβ4GlcβCer.

Previously the inventors have also disclosed type 2 lactosamine binding epitope “neolacto-binding”, -GlcNAcβ3Galβ4GlcNAcβ- for H. pylori. The binding epitope is active on longer chain linear poly-N-acetylactoamines such as NeuNAcα3Galβ4GlcNAcβ3Galβ4GlcNAcβ3Galβ4GlcβCer, however, this binding specificity does not require the presence of sialic acid as the desialylated structures are active (non-sialic acid binding strain) as well as N-glycolyl-neuraminic acid comprising structure NeuNGcα3Galβ4GlcNAcβ3Galβ4GlcNAcβ3Galβ4GlcβCer (FI 20010118), which is not recognized by the sialic acid binding specificity according to the present invention. When the common neolacto epitope was characterized by not-sialic acid binding H. pylori strains, binding to NeuNAcα3Galβ4GlcNAcβ3Galβ4GlcβCer was also not observable (FI20010118).

The sialic acid binding specificity is less common than the neolacto-binding specificity, but the sialic acid binding specificity is probably inflammation related and present in fresh pathologic isolates (Mahdavi et al., 2002). The branching of polylactosamine chain can prevent binding to neolacto epitope but not to the high affinity sialic acid receptor according to the invention (FI20010118).

The prior art has also described a H. pylori binding protein called HPNAP with potential function in neutrophil activation. The present data shows that NAP-protein with sialic acid binding activity is not involved in inhibition of H. pylori binding to sialylated structures. The potential function of the sialic acid binding of NAP-protein also including binding to NeuNAcα3Galβ4GlcNAcβ3Gal-type structures including longer polylactosamines may be related to interactions of H. pylori and neutrophils but its specificity and biological importance is not clear (Teneberg et al., 1997, Teneberg et al., 2000).

The prior art has also described non-sialylated type one epitopes: lactosylceramide Galβ4GlcβCer, gangliotetraosylceramide, Galβ3GalNAcβ4LacβCer, (Lingwood et al., 1992), lacto-structures comprising terminal Galβ3GlcNAcβ- (Teneberg et al. 2002) and Lewis b Fucα2Galβ3(Fucα4)GlcNAcβ- (Borén et al, 1993). These are based on different carbohydrate backbones: lactose, type 1 N-acetyllactosamines and ganglio-core and are clearly different in primary and three-dimensional structures from the sialylated lonrger type 2 N-acetyllactosamines according to the present invention. Moreover the references sited indicates that there are clearly specific strains for the different receptors and in the case of the sialic acid (sLex) binding specificity Mahdavi et al. 2002) and Lewis b-binding the very specific receptor proteins have been actually characterized (Ilver D., et al. 1998).

### BRIEF DESCRIPTION OF THE DRAWINGS

FIG. 1. Negative ion FAB mass spectrum of the ganglioside from human erythrocytes. Above the spectrum is a simplified formula for interpretation, representing the species with sphingosine and non-hydroxy 24:0 fatty acid. The analysis was done as described under “Experimental procedures”. Two spectra were collected, one using an acceleration voltage of 10 kV (A), and a second using an acceleration voltage of 8 kV (B). For spectrum A a range of 100-2400 mass units was scanned, while for spectrum B, a range of 2000-3200 mass units were scanned and data was collected for 2.4 min.

FIG. 2. Electron ionisation mass spectrum of the permethylated ganglioside from human erythrocytes. Above the spectrum is a simplified formula for interpretation, representing the species with sphingosine and non-hydroxy 24:0 fatty acid. The analysis was done as described under “Experimental procedures”. The spectrum was recorded at 380° C. The peak at m/z 354 is due to a contaminant.

FIG. 3. Mass spectrum obtained by electrospray ionisation and collision induced dissociation of the permethylated ganglioside from human erythrocytes. Above the spectrum is a simplified formula for interpretation, representing the species with sphingosine and non-hydroxy 24:0 fatty acid. The analysis was done as described in the “Experimental procedures” section.

FIG. 4. Electron ionisation mass spectrum of the permethylated and reduced ganglioside from human erythrocytes. Above the spectrum is a simplified formula for interpretation, representing the species with sphingosine and non-hydroxy 24:0 fatty acid. The analysis was done as described under “Experimental procedures”. The spectrum was recorded at 340° C.

FIG. 5. Proton NMR spectrum at 600 MHz of the ganglioside from human erythrocytes (30° C.). The sample was dissolved in dimethyl sulphoxide/D₂O (98:2, by volume) after-deuterium exchange. The broad peak(s) centred around 4.8 ppm (indicated by an *) represents a contaminant of unknown origin.

FIG. 6. Binding of H. pylori to NeuAc- and NeuGc-terminated gangliosides. Chemical detection by anisaldehyde (A), and autoradiograms obtained by binding of ³⁵S-labeled H. pylori strains CCUG 17874 (B) and J99 (C). The gangliosides were separated on aluminum-backed silica gel plates, using chloroform/metanol/0.25% KCl in water (50:40:10, by volume) as solvent system, and the binding assay was performed as described under “Experimental procedures”. The lanes were: NeuAcα3Galβ4GlcNAcβ3Galβ4GlcNAcβ3Galβ4Glcβ1Cer (NeuAc-neolactohexaocylceramide) of human hepatoma, 2 μg (lane 1); NeuGcα3Galβ4GlcNAcβ3Galβ4GlcNAcβ3Galβ4Glcβ1Cer (NeuGc-neolactohexaocylceramide) of rabbit thymus, 2 μg (lane 2); NeuAcα3Galβ4GlcNAcβ6 (NeuAcα3Galβ4GlcNAcβ3)Galβ4GlcNAcβ3Galβ4Glcβ1Cer (NeuAc-G-10 ganglioside) of human erythrocytes, 2 μg (lane 3); NeuGcα3Galβ4GlcNAcβ6 (NeuGcα3Galβ4GlcNAcβ3)Galβ4GlcNAcβ3Galβ4Glcβ1Cer (NeuGc-G-10 ganglioside)of bovine erythrocytes, 2 μg (lane 4); Galα3Galβ4GlcNAcβ6 (NeuGcα3Galβ4GlcNAcβ3)Galβ4GlcNAcβ3Galβ4Glcβ1Cer of bovine erythrocytes, 2 μg (lane 5); Galα3(Fucα2)Galβ4GlcNAcβ6 (NeuAcα3Galβ4GlcNAcβ3)Galβ4GlcNAcβ3Galβ4Glcβ1Cer (G9-B ganglioside) of human erythrocytes, 2 μg (lane 6). Autoradiography was for 12 h.

FIG. 7. Comparison of binding of H. pylori strains CCUG 17874, J99. Chemical detection by anisaldehyde (A). Autoradiograms obtained by binding of ³⁵S-labeled H. pylori strain CCUG 17874 (B), and strain J99 (C).

The gangliosides were separated on aluminum-backed silica gel plates, using chloroform/metanol/0.25% KCl in water (50:40:10, by volume) as solvent system, and the binding assays were performed as described in the “Experimental procedures” section. The lanes were: gangliosides of human neutrophil granulocytes, 20 μg (lane 1); NeuGcα3Galβ4GlcNAcβ3Galβ4GlcNAcβ3Galβ4Glcβ1Cer (NeuGc-neolactohexaocylceramide) of rabbit thymus, 2 μg (lane 2); NeuAcα3Galβ4GlcNAcβ3Galβ4GlcNAcβ3Galβ4Glcβ1Cer (NeuAc-neolactohexaocylceramide) of human hepatoma, 1 μg (lane 3); NeuAcα3Galβ4GlcNAcβ6 (NeuAcα3Galβ4GlcNAcβ3)Galβ4GlcNAcβ3Galβ4Glcβ1Cer (NeuAc-G-10 ganglioside) of human erythrocytes, 1 μg (lane 4); Galα3(Fucα2)Galβ4GlcNAcβ6 (NeuAcα3 Galβ4GlcNAcβ3)Galβ4GlcNAcβ3Galβ4Glcβ1Cer (G9-B ganglioside) of human erythrocytes, 1 μg (lane 5); Galα3(Fucα2)Galβ4GlcNAcβ3Galβ4Glcβ1Cer (B6 type 2 hexaglycosylceramide) of human erythrocytes, 4 μg (lane 6); Galβ3GalNAcβ4Galβ4Glcβ1Cer (gangliotetraosylceramide) of mouse feces, 4 μg (lane 7). Autoradiography was for 12-24 h.

FIG. 8. Binding of H. pylori to serial dilutions of gangliosides. (A) Autoradiogram obtained by binding of H. pylori strain CCUG 17874 using the chromatogram binding assay. Lanes 1-7 were serial dilutions (1-100 pmole) of NeuAcα3Galβ4GlcNAcβ3Galβ4Glcβ1Cer (NeuAcα3-neolactotetraosylceramide), NeuAcα3Galβ4GlcNAcβ3Galβ4GlcNAcβ3Galβ4Glcβ1Cer (NeuAcα3-neolactohexaocylceramide), and NeuAcα3Galβ4GlcNAcβ3Galβ4GlcNAcβ3Galβ4GlcNAcβ3Galβ4Glcβ1Cer (NeuAcα3-neolactooctaocylceramide), and lane 8 was NeuAcα3Galβ3(Fucα4)GlcNAcβ3Galβ4Glcβ1Cer (sialyl-Le^{a} hexaglycosylceramide), 1 mmole. The binding assay was done as described under “Experimental procedures”. The results from one representative experiment out of three is shown. (B) Quantification of binding by densitrometry. The autoradiogram in (A) was analyzed using the NIH Image program.

FIG. 9. Binding of H. pylori to serial dilutions of gangliosides. (A) Autoradiogram obtained by binding of H. pylori strain 17874 using the chromatogram binding assay. The lanes were: serial dilutions of NeuAcα3Galβ4GlcNAcβ3Galβ4GlcNAcβ3Galβ4Glcβ1Cer (NeuAc-neolactohexaocylceramide), (10-100 pmole) (lanes 1-5); serial dilutions of NeuAcα3Galβ4GlcNAcβ3Galβ4(Fucα3)GlcNAcβ3Galβ4Glcβ1Cer (VIM-2 ganglioside), (10-100 pmole) (lanes 6-10); serial dilutions of NeuAcα3Galβ4(Fucα3)GlcNAcβ3Galβ4(Fucα3)GlcNAcβ3Galβ4Glcβ1Cer (NeuAc-dimeric-Le^{x} ganglioside), (10-100 pmole) (lanes 11-14); NeuAcα3Galβ4(Fucα3)GlcNAcβ3Galβ4Glcβ1Cer (sialyl-Le^{x} hexaglycosylceramide), 1 mmole (lane 15). The binding assay was done as described under “Experimental procedures”. The results from one representative experiment out of three is shown. (B) Quantification of binding by densitrometry. The autoradiogram in (A) was analyzed using the NIH Image program.

FIG. 10. Binding of H. pylori to serial dilutions of gangliosides. (A) Autoradiogram obtained by binding of H. pylori strain CCUG 17874 to serial dilutions (1-100 pmole) of NeuAcα3Galβ4(Fucα3)GlcNAcβ3Galβ4Glcβ1Cer (NeuAc-Le^{x}), NeuAcα3Galβ4GlcNAcβ3Galβ4GlcNAcβ3Galβ4Glcβ1Cer (NeuAcα3-neolactohexaocylceramide), NeuAcα3Galβ4GlcNAcβ6(NeuAcα3Galβ4GlcNAcβ3)Galβ4GlcNAcβ3Galβ4Glcβ1Cer (NeuAc-G-10 ganglioside) and Galα3(Fucα2)Galβ4GlcNAcβ6(NeuAcα3Galβ4GlcNAcβ3)Galβ4GlcNAcβ3Galβ4Glcβ1Cer (G9-B ganglioside) using the chromatogram binding assay. The binding assay was done as described under “Experimental procedures”. The results from one representative experiment out of three is shown. (3) Quantification of binding by densitrometry. The autoradiogram in (A) was analyzed using the NIH Image program. For comparison, the result densitometry of the binding of ¹²⁵I-labeled cholera toxin B-subunits (CTB) to dilutions of the GM1 ganglioside on a thin-layer chromatogram is included in (A).

FIG. 11 Detection level of granulocyte gangliosides on TLC plates using radiolabeled H. pylori. The plates were developed in C/M/0.25% KCl in water, 50:40:10, and visualized by spraying with anisaldehyde (Anis) or by overlay with ³⁵S-labeled H. pylori, CCGU 17874. Lanes 1-10, two-fold dilutions of granulocyte gangliosides (total 12 μg in Lane 1). Lane 11, bovine brain gangliosides, 2 μg (mixture of GM1, GD1a, GD1b and GT1b); S-3PG and S-6PG stand for NeuAcα3Galβ4GlcNAcβ3Galβ4GlcCer and NeuAcα6Galβ4GlcNAcβ3Galβ4GlcCer, respectively. Annotations 7s and 8s indicate chromatographic regions containing gangliosides with 7 and 8 monosaccharides per molecule.

FIGS. 12A, 12B, 12C, 12D, 12E, 12F, 12G, 12H, 12I, 12J, and 12K. Negative ion FAB spectra of S-3PG derivatised at the —CH₂CH₂CH₂OH and —COOH groups of the sialic acid. 12A, S-3PG, unmodified; 12B, oxidized/reduced; 12C, After oxidation and derivatization with methylamine; 12D, After oxidation and derivatization with ethanolamine. 12E, reduced at —COOH; 12F, free amide; 12G, Methylamide; 12H. Ethylamide; 12I, Propylamide; 12J, benzylamide; 12K, Octadecylamide

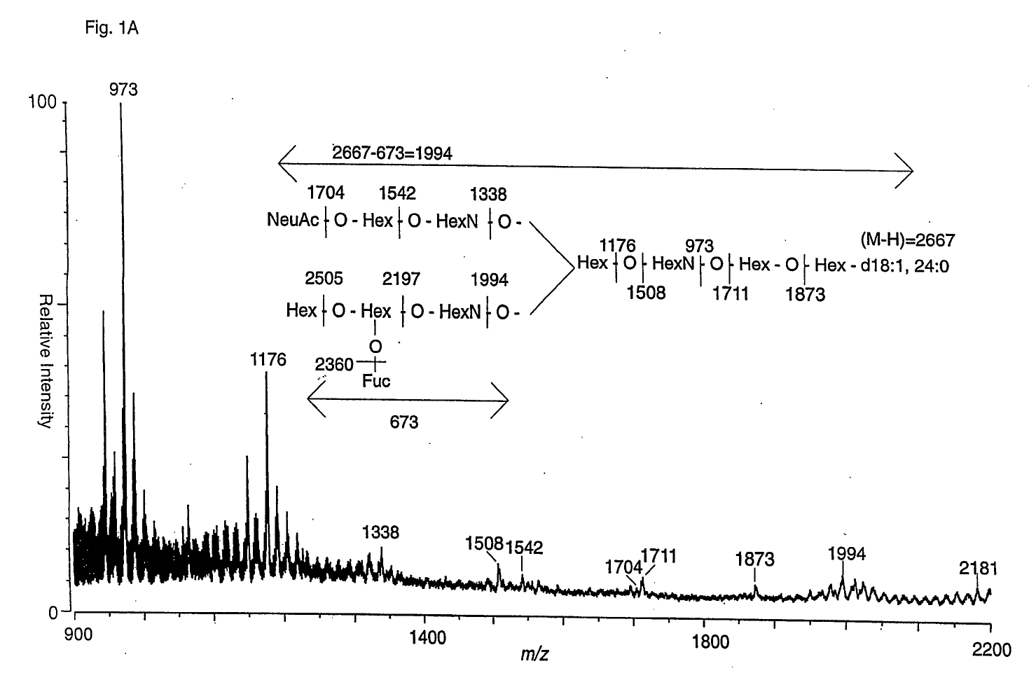

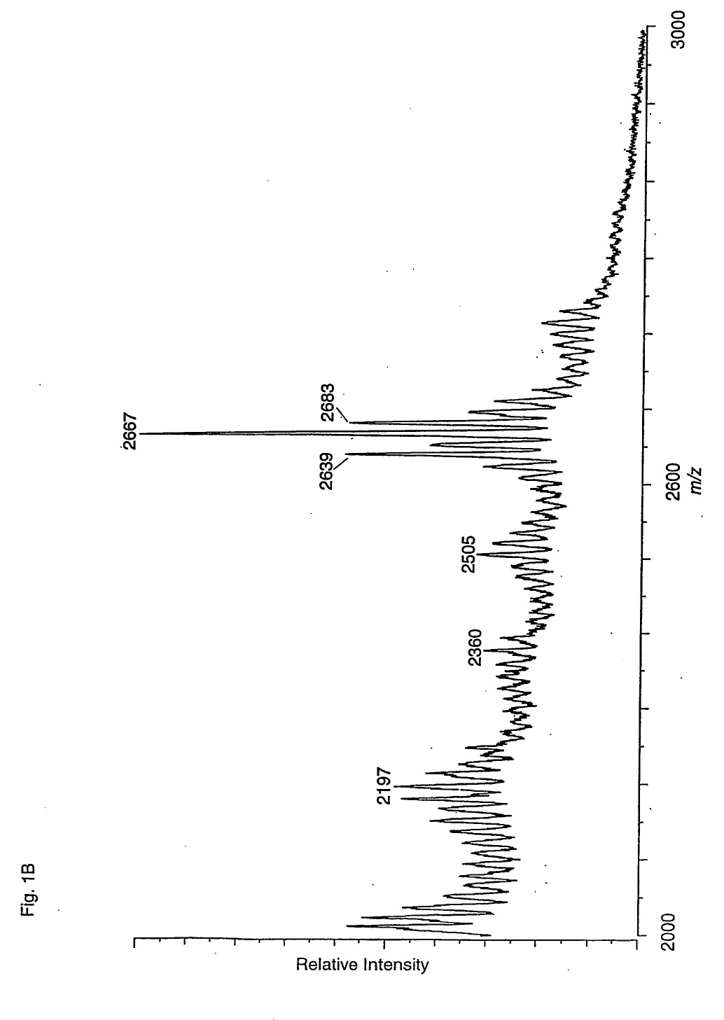

FIG. 13. Example of binding of H. pylori (CCGU 17874) to modified S-3PG. Glycolipids were separated on TLC plates and visualized with anisaldehyde (Anis, left plate) or with with ³⁵S-labeled bacterium (H. pylori, right plate). For chromatographic conditions see FIG. 1. Lane 1, S-3PG. This lane also contains trace amounts of longer members of the binding series; Lane 2, ethylamide of S-3PG; Lane 3, propylamide of S-3-PG; Lane 4, benzylamide of S-3-PG; Lane 5, oxidized/reduced S-3-PG, see Table 3; Lane 6, positive control for H. pylori; Lane 7, bovine brain gangliosides (from top: GM1, GD1a, GD1b, GT1b); Lane 8, mixture of sulfatides; Lane 9, mixture of five sugar-containing gangliosides from rabbit thymus; Lane 10, mixture of complex gangliosides prepared from human granulocytes.

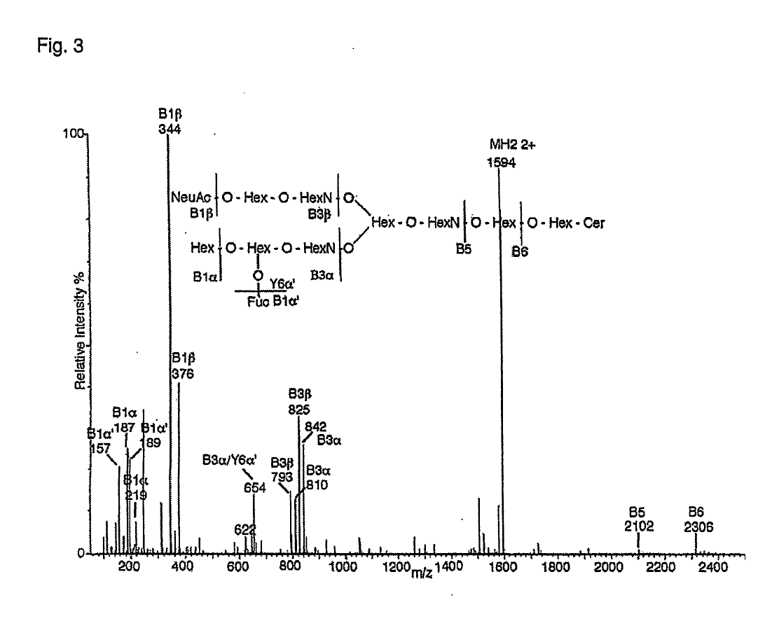

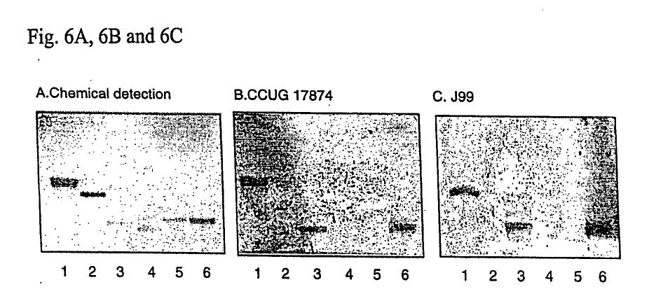

FIG. 14. Binding of H. pylori (CCGU 17874 strain) to neoglycolipids on TLC plates. For chromatographic conditions see FIG. 1. Left plate was stained for carbohydrates by anisaldehyde and the right plate was overlaid with ³⁵S-labeled H. pylori. Lane 1, S-3PG. Like in FIG. 3, the SPG preparation contains trace amounts of longer members of the binding series; Lane 2, neoglycolipids formed from NeuAcα3Galβ4GlcNAcβ3Galβ4Glc; Lane 3, neoglycolipids formed from NeuAcα3Galβ3GlcNAcβ3Galβ4Glc; Lane 4, neoglycolipids formed from NeuAcα6Galβ4GlcNAcβ3Galβ4Glc; Lane 5, neoglycolipids formed from Galβ3(NeuAcα6)GlcNAcβ3Galβ4Glc; Lane 6, bovine brain gangliosides (from top: GM1, GD1a, GD1b, GT1b). Arrows in lane 2 indicate hexadecylaniline-derivative (lower band) and neoglycolipid with branched lipid chain (upper double band). The marked fractions in lane 2 and the corresponding fractions in lanes 3-5 were scraped off and tested by mass spectrometry, see FIG. 6.

FIG. 15. Negative ion FAB spectra of neoglycolipids derived from NeuAcα3Galβ4GlcNAcβ4Galβ4Glc. Panel A, hexadecylaniline derivative; Panel B, neoglycolipid with branched lipid part.

FIG. 16. Example of EI MS (electron ionization mass spectrometry) of permethylated polyglycosylceramides showing fragment ions corresponding to NeuAc and the reduced NeuAc.

FIG. 17. Binding of H. pylori(032 strain) to derivatized PGCs on TLC plates. Left plate was stained for carbohydrates by anisaldehyde and the right plate was overlaid with ³⁵S-radiolabeled H. pylori. Lanes 1 and 2, underivatized PGCs of human erythrocytes; Lane 3, reduced PGCs (COOH->CH₂OH); Lane 4, bovine brain gangliosides (mixture of GM1, GD1a, GD1b, GT1b); Lane 5, S-3PG. (Note that bacteria 032 från broth do not bind to S-3PG which represent the linear structure).

### DETAILED DESCRIPTION OF THE INVENTION

The present invention shows that several linear and branched NeuNAcα3-poly-N-acetyllactosamine structures can serve as high affinity ligands for Helicobacter pylori. The binding is specific for NeuNAcα3 linked to the type lactosamine Galβ4GlcNAc. The present invention is directed to larger polylactosamines having higher binding activity than the terminal trisaccharide epitope. When considering the linear polylactosamine structures NeuNAcα3Galβ4GlcNAcβ3Galβ4GlcNAcβ3Galβ4GlcβCer and NeuNAcα3Galβ4GlcNAcβ3Galβ4GlcNAcβ3Galβ4GlcNAcβ3Galβ4GlcβCer it is clear that the presentation of the terminal NeuNAcα3Galβ4GlcNAc is effective on β3Galβ4GlcNAc and larger polylactosamine β3Galβ4GlcNAcβ3Galβ4GlcNAc. The results also indicate that the terminal structure β3-linked on Gal or lactose are useful minimal epitopes.

General Formula of Novel High Affinity Inhibitors of H. pylori

The present invention is specifically directed to high affinity Helicobacter pylori binding oligosaccharide sequences according to the Formula 1:
R₁Galβ4GlcNAcβ3{(R₂Galβ4GlcNAcβ6)}ₛ₁Gal[β4GlcNAcβ3Gal]ₛ₂{β4Glc[NAc]ₛ₃}ₛ₄
wherein R1 and R2 are independently nothing, or terminal mono- or oligosaccharides substituents with the proviso that at least one of the substituents is NeuNAcα3 or NeuNAcα3Galβ4GlcNAcβ3. Integers s1, s2, s3 and s4 are independently 0 or 1, indicating the presence or absence of the structures in [ ] or in { }. The possible branch in the structure is marked as ( ). The present invention is also directed to structural analogs, especially conformational analogs, or derivatives of said oligosaccharide sequence having binding activity to Helicobacter pylori
Structure of Potential Non-Sialylated Branch Structure

Preferably R1 or R2, when not being NeuNAcα3, indicates terminal substituents linked to position 2 and/or 3 of the terminal Gal according to Formula 2
Hex[NAc]ₜ₁α/β3[(DeoxyHexα2)]ₜ₂
wherein Hex is preferably Gal or Glc. Integers t1 and t2 are independently 0 or 1. α/β means that the linkage is either α or β.

Preferably non-sialylated R1 or R2 is a structure selected from the group consisisting of Galα3, GalNAcα3, Fucα2, Galα3(Fucα2), GalNAcα3(Fucα2), NeuNAcα3Galβ4GlcNAcβ3, Galβ4GlcNAcβ3, GlcNAcβ3Galβ4GlcNAcβ3, GlcNAcα3, GlcNAcβ3, GalNAcβ3, Galβ3, Glcβ3, and Glcα3. More preferably the structure is selected from the group of blood group antigen like structures: Galα3, GalNAcα3, Fucα2, Galα3(Fucα2), and GalNAcα3(Fucα2).

Preferred Branched Structures

In a preferred embodiment s1 is 1 and both R1 and R2 are selected from the group consisting of NeuNAcα3 or NeuNAcα3Galβ4GlcNAcβ3. The preferred poly-N-acetylactosamine structures include oligosaccharide sequences

NeuNAcα3LacNAcβ3(NeuNAcα3LacNAcβ6)LacNAcβ3LacNAc

NeuNAcα3LacNAcβ3(NeuNAcα3LacNAcβ3LacNAcβ6)LacNAc

NeuNAcα3LacNAcβ3LacNAcβ3(NeuNAcα3LacNAcβ6)LacNAc

NeuNAcα3LacNAcβ3(NeuNAcα3LacNAcβ6)LacNAcβ3Lac

NeuNAcα3LacNAcβ3(NeuNAcα3LacNAcβ6)LacNAcβ3Gal

NeuNAcα3LacNAcβ3(NeuNAcα3LacNAcβ6)Lac

NeuNAcα3LacNAcβ3(NeuAcα3LacNAcβ6)LacNAc

NeuNAcα3LacNAcβ3(NeuNAcα3LacNAcβ6)Gal

The oligosaccharide sequences according to the present invention may be further presented as branched poly-N-acetyllactosamines, for example
NeuNAcα3LacNAcβ3(NeuNAcα3LacNAcβ6)LacNAcβ3(NeuNAcα3LacNAcβ6)LacNAc
wherein LacNAc indicates N-acetyllactosamine, Galβ4GlcNAc, and Lac is lactose, Galβ4Glc, the two sequences below indicate the same structures: NeuNAcα3Galβ4GlcNAcβ3(NeuNAcα3Galβ4GlcNAcβ6)Galβ4Glc NeuNAcα3LacNAcβ3(NeuNAcα3LacNAcβ6)Lac
Preferred Long Chain Lactosamine Epitopes

When s1 is 0 the Formula 1 describes linear sialylpolylactosamines such as

NeuNAcα3LacNAcβ3LacNAcβ3LacNAc

NeuNAcα3LacNAcβ3LacNAcβ3Lac

NeuNAcα3LacNAcβ3LacNAcβ3Gal

NeuNAcα3LacNAcβ3LacNAc

And shorter minimal epitopes:

NeuNAcα3LacNAcβ3Lac

NeuNAcα3LacNAcβ3Gal

Preferred Analog Structures

The present invention shows that the glycerol tail of the NeuNAc residue does not tolerate modifications produced by the oxidation and reduction. Acetyl group of NeuNAc does not tolerate much changes, thus glycolyl-, propyl-, or deacetylated amine analogs are weakly active or not active at all. Also the variants in which the carboxylic acid group of the sialic acid is reduced to alcohol are not active. The similar specificities were shown with small pentasaccharide epitopes and branched large polyglycosylceramides. Positioning of the sialic acid residue is also important, disialic acid or NeuNAcα6-structures were not active. Futhermore the terminal N-acetylactosamine to which the NeuNAc-residue is α3-linked should be type two N-acetylactosamine Galβ4GlcNAc, while type 1 N-acetyllactosamine, Galβ3GlcNAc, or asialo-ganglioside terminals, Galβ3GalNAc are not accepted. The data allows effective design of tolerable analogs and derivatives avoiding the non-active structural features.

The present invention is especially directed to analogs of the structures according to the Formula 1 wherein at least one of N-acetylactosamine residues have been replaced by type 2 N-acetyllactosamine analogous structure or structures, preferably by lactose residues according to Formula 3
R₁Galβ4Glc[NAc]ᵤ₁β3{(R₂Galβ4Glc[NAc]ᵤ₂β6)}ₛ₁Gal{β4Glc[NAc]ᵤ₃β3Gal}ₛ₂{β4Glc[NAc]ₛ₃}ₛ₄
wherein R1 and R2 are independently nothing or terminal mono- or oligosaccharides substituents with the proviso that at least one of the substituents is NeuNAcα3 or NeuNAcα3Galβ4Glc[NAc]ᵤ₄β3. Integers s1, s2, s3 and s4 are independently 0 or 1, indicating the presence or absence of the structures in [ ] or in { }. Integers u1, u2, u3, and u4 are independently 0 or 1, indicating the presence of absence of the N-acetyl groups in the non-reducing end terminal or midchain lactosamine residues with the proviso that at least one of the integers present is 0. Type 2 N-acetyllactosamine analogous structures described herein include Galβ4Glc2-X structures in which carbon 2 of Glc-ring is linked to group X, which is preferably —NH₂ (GalβGlcN-analog) or N-alkyl (Galβ4GlcN-alkyl) or N-alkanoyl. A preferrred alkanoyl is propanoyl. Carbon 2 may also be derivatized by an O-ester such as O-acetyl or O-ether such as O-methyl. Especially disialylated and linear monosialylated structures are preferred:
NeuNAcα3Lac[NAc]ᵤ₁β3(NeuNAcα3Lac[NAc]ᵤ₂β6)Lac[NAc]ᵤ₃β3Gal {β4Glc[NAc]ₛ₃}ₛ₄
NeuNAcα3Lac[NAc]ᵤ₁β3(NeuNAcα3Lac[NAc]ᵤ₂β3Lac[NAc]ᵤ₃β6)Gal{β4Glc[NAc]ₛ₃}ₛ₄
NeuNAcα3Lac[NAc]ᵤ₁β3Lac[NAc]ᵤ₂β3(NeuNAcα3Lac[NAc]ᵤ₃β6)Gal{β4Glc[NAc]ₛ₃}ₛ₄
NeuNAcα3Lac[NAc]ᵤ₁β3Lac[NAc]ᵤ₂β3Gal{β4Glc[NAc]ₛ₃}ₛ₄

The invention is also directed to analogs according to structure
NeuNAcα3Lac[NAc]ᵤ₁β3Lac[NAc]ᵤ₂
with the proviso that when u2 is 0 then u1 is also 0.
Fucosylated Analogs

As discussed above the fucoslated structures such as sLex and sdiLex have been studied separately. The present invention shows that the same binding specificity is involved with both fucosylated and non-fucosylated sialylα3-N-acetyllactosamines. It is realized that the lactosamine anologs of the poly-Nacetyllactosamine described above, especially the lactose comprising analogs would also be active when α3-fucosylated to Glc and (if also present) to GlcNAc residues. Such analogs would combine cheaper backbone structures with higher affinity induced by the fucose residues. The present invention is directed to the sialylated and fucosylated structures as such.

The most preferred facosylated structures are according to the formula 4
R₁Galβ4[(Fucα3)]ₜ₁Glc[NAc]ᵤ₁β3 {(R₂Galβ[(Fucα3)]ₜ₂Glc[NAc]ᵤ₂β6)}ₛ₁Gal{β4[(Fucα3)]ₜ₃Glc[NAc]ᵤ₃β3Gal}ₛ₂{β4[(Fucα3)]ₜ₄Glc[NAc]ₛ₃}ₛ₄
wherein R1 and R2 are independently nothing or terminal mono- or oligosaccharides substituents with the proviso that at least one of the substituents is NeuNAcα3 or NeuNAcα3Galβ4[(Fucα3)]ₜ₅Glc[NAc]ᵤ₄β3. Integers s1, s2, s3, and s4 are independently 0 or 1, indicating the presence or absence of the structures in [ ] or in { }. Integers u1, u2, u3, and u4 are independently 0 or 1, indicating the presence of absence of the N-acetyl groups in the non-reducing end terminal or midchain lactosamine residues with the proviso that at least one of the integers present is 0. Integers t1, t2, t3, t4 and t5 are independently 0 or 1, indicating the presence or absence of the Fucα3-branch-structures in [ ]. Especially disialylated and linear monosialylated structures are preferred.

The present invention is also directed to non-fucosylated and fucosylated analog structures comprising N-acetyllactosamine analogue at non-reducing position. Especially, analogs comprising lactose are preferred, wherein at least on of u1, u2 or u3 is 0, more preferably two of the variables are 0 and most preferably all three are 0. In a preferred embodiment the analog is branched and more preferably disialylated. Separately, pentasaccharide, hexasaccharide and heptasaccharide structures are preferred as analog substances according to the invention.

Minimal Epitopes Giving Better Presentation of NeuNAcα3Galβ4Glc(NAc)

The structure close to ceramide is sterically more restricted in the TLC binding assay. As a free monovalent inhibitor or as polyvalent conjugate the oligosaccharide sequences NeuNAcα3Galβ4GlcNAcβ3Galβ and NeuNAcα3Galβ4GlcNAcβ3Galβ4Glc and especially NeuNAcα3Galβ4GlcNAcβ3Galβ4GlcNAc are more active than the sialylactose or NeuNAcα3Galβ4GlcNAc. The use of neoglycolipid structure demonstrated that reductively aminated structure NeuNAcα3Galβ4GlcNAcβ3Galβ4Glc-hexadecylaniline is active even though the reducing end glucose is not in ring form after the reductive conjugation. The activity was similar to the glycolipid structure NeuNAcα3Galβ4GlcNAcβ3Galβ4GlcβCer when the trisaccharide glycolipid NeuNAcα3Galβ4GlcβCer (GM3) was inactive.

The present invention is specifically directed to functional foods, especially infant foods including infant formulas and food additives comprising the added or enriched sialylated polylactosamines according to the present invention which are present in human and animal milks. Especially preferred structures for the food uses include one or several oligosaccharides selected from the group NeuAcα3Galβ4GlcNAcβ3Galβ4Glc, NeuNAcα3Galβ4GlcNAcβ3Galβ4GlcNAcβ3Galβ4Glc, NeuNAcα3Galβ4GlcNAcβ3(NeuNAcα3Galβ4GlcNAcβ6)Galβ4Glc and more preferably the structures for functional foods include NeuAcα3Galβ4GlcNAcβ3Galβ4Glc and/or NeuNAcα3Galβ4GlcNAcβ3Galβ4GlcNAcβ3Galβ4Glc and most preferably NeuAcα3Galβ4GlcNAcβ3Galβ4Glc. The present invention is further directed to oligosaccharide composition comprising the three oligosaccharides or any combinations of two oligosaccharides selected from the above group in essentially pure form (essentially pure oligosaccharide fraction comprise at least 80 carbohydrate mass % of the desired oligosaccharide or oligosaccharides, more preferably the essentially pure fraction comprises at least 90% and most preferably at least 95% of the desired oligosaccharides) and use of the compositions in preparation of functional foods or pharmaceutical or therapheutic compositions. The present invention is further directed to the use of the oligosaccharide sequences in chewing gums and various consumer products.

In another embodiment of the invention it is also preferred to have a sialylated oligosaccharide from the above group or 2 or 3 of the saccharides as an essentially pure mixture with one or several oligosaccharides selected from the group consisting of human milk or animal milk saccharides such as sialyl lactose(s), lactose, lacto-N-neotetraose, para-lacto-N-neotetraose, lacto-N-neohexaose and α3-fucosylated derivates thereof. A preferred sialic oligosaccharide mixture contains NeuNAcα3Lac, NeuNAcα3LacNAcβ3Lac and NeuNAcα3(LacNAcβ3)₂Lac or more preferrably such mixture contains only the two first mentioned.

The present invention is especially directed to minimal higher affinity receptor oligosaccharide sequence according to the formula 5
NeuNAcα3Galβ4GlcNAcβ3Gal
with the proviso that the structure is not presented as a pentasaccharide glycolipid NeuNAcα3Galβ4GlcNAcβ3Galβ4GlcβCer. More preferably the oligosaccharide sequence is not linked to ceramide or a hydrophobic aglycon or spacer comprising more than 22 carbon atoms. More preferably the tetrasaccharide sequence is coupled to an aglycon or spacer comprising less than 8 carbon atoms in a hydrophobic structure.

The present invention is also directed to the conformational analogs and derivative of the above structures. Preferably the structural analog or derivative structures have similar or better affinity towards H. pylori. In a preferred class of analogs the analog comprise lactose or another disaccharide epitope having similar conformation with type 2 N-acetyllactosamine. Preferred analog structures include oligosaccharide sequences NeuNAcα3Galβ4Glcβ3Gal. The present invention is specifically directed to substances comprising terminal oligosaccharide sequence NeuNAcα3Galβ4Glcβ3Gal. The lactose based structures are much cheaper to produce chemically than N-acetyllactosamine structures.

The present invention is especially directed to minimal higher affinity receptor oligosaccharide sequence according to the formula 6
NeuNAcα3Galβ4GlcNAcβ3Galβ4Glc(NAc)ₘ
wherein m is 0 or 1 with the proviso that the structure is not presented as a pentasaccharide glycolipid NeuNAcα3Galβ4GlcNAcβ3Galβ4GlcβCer. More preferably the oligosaccharide sequence is not linked to ceramide or a hydrophobic aglycon or spacer comprising more than 22 carbon atoms. More preferably the tetrasaccharide sequence is coupled to an aglycon or spacer comprising less than 8 carbon atoms in a hydrophobic structure. The minimal higher affinity sequences are especially useful in polyvalent conjugates and as free saccharides.

The present invention is also directed to the conformational analogs and derivative of the structures. Preferably the structural analog or derivative structures have similar or better affinity towards H. pylori. In a preferred class of analogs the analog comprise lactose or another disaccharide epitope having similar conformation with type 2 N-acetyllactosamine. Preferred analog structures include oligosaccharide sequences NeuNAcα3Galβ4Glcβ3Galβ4Glc(NAc)ₘ wherein m is 0 or 1. The present invention is specifically directed to substances comprising terminal oligosaccharide sequence NeuNAcα3Galβ4Glcβ3Galβ4Glc or NeuNAcα3Galβ4Glcβ3Galβ4GlcNAc. The lactose based structures are much cheaper to produce chemically than N-acetylactosamine structures.

Hydrophilic Oligosaccharide Substances.

The present invention is specifically directed to hydrophilic oligosaccharide substances. The inventors found out that the binding to the novel high affinity oligosaccharide sequences according to the invention was independent of ceramide structure present. Previously, it has been known that part of bacterial and especially H. pylori, bindings toward glycolipds are dependent of ceramide structures and part of these are not. The present invention is directed to a useful high-affinity oligosaccharide binding independent of the lipid part. The data in the examples show that H. pylori binds to specific high affinity oligosaccharide sequences independent of the ceramide structures.

Previously a ceramide independent effects have shown for shorter sialyl-lactose structures/sialyllactosamine structures which are trisaccharides, or larger fucosylated sialyllactosamines. The binding specificities with larger sialyl-polylactosamines according to the published seem to be different from the trisacccharide-specificity as specificity for the shorter epitopes is not present in Helicobacter pylori grown under certain conditions. The present invention, directed to this high affinity binding specificity or mode of H. pylori, is clearly different from the small saccharide experiments. The present invention is further directed to inhibition of H. pylori binding with the substances according to the invention and methods to remove H. pylori from the patient by the substances according to the invention. The prior art has also indicated different high affinity effects specifically related to the fucosylated sialylactosamines, sialyl-Lewis x type structures.

The ceramide based structures are not in general useful for single molecule based therapies as the ceramides have natural tendency to associate with cellular membranes. If the ceramide compound aimed for inhibition of the adhesion would be used for inhibition of the adhesion, there would be a risk for adherence of the lipids to the tissue and increased binding to the infected tissue instead of the inhibition. Moreover the ceramide based structures are laborious and relative expensive to synthesize, produce and formulate for any medical application. The glycolipid substances are further known to have antigenic characteristics.

The present invention about the ceramide independent high affinity oligosaccharide sequences is therefore especially useful for various therapeutic approaches for treatment or prevention of H. pylori infections. Instead of the use of the glycolipids the present invention is directed to use of hydrophilic oligosaccharides sequences. The hydrophilic oligosaccharide sequence according to the present invention contains the high affinity oligosaccharide sequence according to the invention and optionally hydrophilic structure. In a specific embodiment the oligosaccharide sequence is the corresponding oligosaccharide. In specific embodiment the oligosaccharide sequence is preferably used for functional food applications, especially for infant formula application according to the invention. The additional useful aspect with the use of natural human milk or animal milk oligosaccharides is the possibility to use natural oligosaccharides with good safety profile and possible multiple beneficial aspect with regard to treatment of infections. The hydrophilic structure is preferably linked to the reducing end of the oligosacharide sequence and it is
1) a non-hydrophobic aglycon structure increasing monovalent binding of oligosaccharide sequence or
2) a non-hydrophobic aglycon structure being a spacer linking the oligosaccharide sequence to a polyvalent or a multivalent carrier or a hydrophilic monovalent carrier.

The non-hydrophobic aglycon structure means a structure which is substancially less hydrophobic that the natural ceramide structures. This can be achieved by organic carbon based structures comprising less carbon atoms or structures comprising substantially larger ratio hydrophilic groups such as oxygen atoms or nitrogen atoms in relation to aliphatic —CH₂— structures than in natural glycolids. Preferably the ratio of hydrophilic groups to —CH₂— structures is at least 1 to 4, more preferably at least 1 to 3, even more preferably at least 1 to 2 and most preferably about 1 to 1 and in a specific more preferred embodiment less than 1 to 1. Preferably the aglycon comprises not more than one aliphatic structure comprising more than 3 CH₂— structures linked to each other. The preferred aglycon structure contains preferably less than 23 carbon atoms, more preferably less than 15 and even more preferably less than 10 carbon atoms. In preferred embodiments the aglycon structure contains a ring structure, preferably a six atom ring structure or five atom ring structure and even more preferably a six membered ring structure and/or at least one hydroxyl group more preferably at least two hydroxyl groups and in a preferedd embodiment at least three hydroxyl groups and/or at least one amide structure, preferably an acetamido (also called as N-acetyl)-structure or a structure mimicking an aceamido-structure of a GlcNAc-residue. More preferably the aglycon comprises a ring structure and a hydroxyl group or a ring structue and an amide structure. Preferred six membered ring includes cyclohexane and bezene-rings and substituted derivatives thereof, and heterocyclic ring structures comprising five carbon atoms and a nitrogen, oxygen or sulphur heteroatom.

When the non-hydrophobic aglycon substance is linked to the reducing end of the oligosaccharide sequence it is preferably aimed for increasing the affinity of the oligosaccharide by increasing the affinity of the oligosaccharide sequence. Especially the non-hydrophobic aglycon substances are preferred for increasing the affinity of monovalent substances, the aglycon substance being a spacer comprising in a preferred embodiment a structure further increasing the affinity of the oligosaccharide sequence. Preferably the aglycon structure contains at least one five atom or six atom ring structure mimicking the reducing end oligosaccharide structure in a longer version of the high affinity oligosaccharide sequence according to the invention, more preferably the aglycon comprises a single five atom of six atom ring structure mimicking a reducing end monosaccharide residue in a one monosaccharide longer oligosaccharide sequence according to the present invention. In a more specific embodiment the aglycon is preferably a mimic of a hexose structure, more preferably a mimick of a D-galactopyranosyl structure when the reducing end monosaccharide residue of the oligosaccharide sequences is GlcNAc or Glc or mimic of D-glucopyranosyl or D-N-acetylglucopyranosyl when the reducing end monosaccharide residue of the oligosaccharide sequence is Gal. More preferably the aglycon mimics an additional reducing end monosaccharide so that it further mimics the glycosidic bond structure between the monosaccharides in a one monosaccharide longer oligosaccharides, preferably the reducing end structure mimicks either Gal to which the oligosaccharide is linked to 3 position, preferably β3-linked, or it mimics Glc or GlcNAc to which the oligosaccharide is linked to 4 position, preferably β4-linked.

The present invention specifically directed hydrophilic oligosaccharides according to invention when the oligosaccharides are free oligosaccharides or linked from the reducing end to a aglycon as described by the invention. Preferably the aglycon is a hydrophilic substance of less than 23 carbon atoms. In a specific embodiment the aglycon is linker to carrier preferably a polyvalent or oligovalent carrier, meaning that oligosaccharide sequence can be linked oligo or polyvalently to the carrier structure.

Preferred aglycon structures further includes small structures comprising one to about 8 carbon atoms more preferably one to 3 and most preferably one or two carbon atoms. The small structures includes for example polyol substances such as reduced monosaccharide units, glycosides simple alcohols such as methanol, ethanol, propanol, isopropanol, glycol, glycolic acid, glycerol, glycerate, carboxylix acid alcohols, aminoacid residues, preferably natural L-aminoacids, more preferably glycosides of serine or threonine. The preferred small aglycon structures further includes carboxylic acid linked to the reducing end preferably derived to to glycosylamide preferred carboxylic acids includes C1-C8 carboxylic acids, preferably C1-C4 carboxylic acids, most preferably acetic acid and even more preferably acetic acid amide bonded to the reducing end of the oligosaccharide sequence. The preferred small structures can be also used spacer, the small spacer structures containing preferably two functional groups are used as spacers.

Preferred spacers structures according to the invention are described for example as hydrophilic spacer structures according to the invention. In a preferred the polyvalent carrier is a polysaccharide and the oligosaccharide is linked to the polysaccharide by a group specifically reactive to reducing aldehyde of the oligosaccharide without need of chemical protection of the oligosaccharide. Preferably the carrier is a hydrophilic molecule and a watersoluble molecule, most preferably the carrier is a polysaccharides such as chitosan, glycogen or starch polymer or oligomer, cyclodextrin, chondroitin, chondroitin sulphate, heparin or hyaluronic acid.

The oligosaccharide structure is preferably β-linked to the aglycon structure.

In a specific embodiments the aglycon structure mimicks the binding interactions of the monosaccharide at the reducing end. To mimick the binding interactions of a monosaccharide residue the aglycon substance may comprise hydrogen bonding structures mimicking the bindings of hydroxyl groups or ring oxygens of a monosaccharide residue or an hydrophilic structure mimicking a hydrophilic interaction with a monosaccharide residue and a hydrophilic aminoacid side chain on protein surface. In a preferred embodiment an aromatic ring structure, more preferably a six membered aromatic ring structure is used for micmicking the binding of the reducing end monosaccharide residue.

The present invention is specifically directed to methods and assays for screening aglycon structures preferred according to the invention using the binding of the oligosaccharide sequences according to the invention to Helicobacter pylori. In a preferred method protein structure of sialic acid binding adhesin is used for design of the aglycon structures. The present invention is further directed to search and design of analogs of the shortest oligosaccharide sequences according to the invention when the reducing end monosaccharide residues is replaced by an aglycon mimicking the recucing end monosaccharide residue as described by the invention. Preferably the present invention is directed to the screening of analogs for NeuNAcα3Galβ4Glcβ3Gal and NeuNAcα3Galβ4GlcNAcβ3Gal, wherein the 3Gal is replaced by the aglycon mimicking the monosaccharide residue or a structure mimicking the binding of 3Gal with H. pylori. Preferably the present invention is directed to the screening of analogs for NeuNAcα3Galβ4Glcβ3Galβ4Glc(NAc)_{0 or 1} and NeuNAcα3Galβ4GlcNAcβ3Galβ4Glc(NAc)_{0 or 1}, wherein the 4Glc(NAc)_{0 or 1} is replaced by the aglycon mimicking the monosaccharide residue or the binding of the monosaccharide residues with H. pylori. The present invention is thus preferably directed to screening of analogs of the oligosaccharide sequences according to the present invention according to the formula
NeuNAcα3Galβ4Glc(NAc)ₛ₁{[β3Gal}ₛ₂-R
s1 and s2 are independly 0 or 1
wherein R is the aglycon mimicking β3Gal or its binding to H. pylori when s2 is 0 and
R is aglycon mimicking β4GlcNAc or β4Glc or binding of β4GlcNAc or β4Glc when s2 is 1. The structures when s2 is 0 are preferred in a specific embodiment as the oligosaccharide part can be produced from cheap natural sources such as from bovine colostrum or waste stream of cheese production.
Preferred Large Poly-N-Acetyllactosamines and Preferred Uses Thereof.

In a specific embodiment the present invention is directed to structures according to the Formula
R₁Galβ4GlcNAcβ3{(R₂Galβ4GlcNAcβ6)}ₛ₁Gal{β4Glc[NAc]ₛ₃}ₛ₄
wherein R1 and R2 are terminal mono- or oligosaccharides substituents so that at least one of the substituents is NeuNAcα3; s1, s3 and s4 are independently integers 0 or 1 indicating presence or absence of the structure in { } or in [ ];
as a non-reducing end terminal sequence, and Helicobacter pylori binding analogs and derivatives thereof, for use as a medicament.

These larger poly-N-acetylactosamine structures are especially preferred when produced from natural sources. Such large poly-N-acetylactosamines can be produces from natural glycoproteins or glycolipids by methods known for release of oligosaccharides including endo-glycosidase methods for release of N-glycans for example by protein N-glycosidases or release of O-linked glycans by digesting the glycoprotein proteases to corresponding glycopeptides. Production of glycopeptides by food acceptable methods, especially protease methods are preferred. Protein linked N-glycans can be also released by beta-elimination methods involving optionally reduction of the reducing end of oligosaccharides or for example hydrazine based release methods known in the art. Oligosaccharides can be released from glycolipids by otsonolysis or by endo-glycosylceramides. The invention is specific embodiment directed to enzymatically released protein linked oligosaccharides or glycopeptides and chemically released protein linked oligosaccharides and peptides and mixtures thereof. Other embodiments are directed to oligosaccharides produced by chemical or enzymatic means from glycolipids. The structures are especially preferred as mixtures produced from natural sources. The present invention is further directed natural or released oligosaccharides or glycopeptides enriched with large poly-N-acetyllactosamines according to the invention.

The present invention is further directed natural glycoprotein fractions enriched with large poly-N-acetyllactosamimes according to the invention. Present invention is further directed to fractions of natural poly-N-acetyllactosamine precursors for enzymatic remodelling of the precursors to structures according to the invention. Numerous animal based protein and lipid materials containing poly-N-acetyllactosamine structures have been published and are known in the art. The present invention is especially directed to food acceptable materials.

In a specific embodiment the present invention is directed to use of the preferred large poly-N-acetyllactosamines according to the invention as glycolipids as micelles for therapy according to the invention or for testing of H. pylori binding. The substances are especially preferred for screening of H. pylori binding when high affinity ligands are searched.

Production of Functional Oligosaccharides by Remodelling Natural Glycans

In a specific embodiment the present invention is directed to remodelling natural glycans, preferably poly-N-acetyllactosamine containing from food sources to functional food carbohydrates. Methods to remodel glycans are described by recent US/WO-patent applications by Neose-company. The present invention is especially directed to desialylation and/or defucosylation of natural food glycoproteins or oligosaccharides or released oligosaccharides. Most preferably the oligosaccharides or glycopeptides are desialylated mild acid treatment. In a preferred embodiment the desialylation is performed by, treatment with food acceptable carboxylic acid and heating to about 80-100 degrees of Celsius as known in the art, preferably by acetic acid. In another embodiment the food material is desialylated by volatile acid, for example hydrochloric acid, which can be removed from heat treatment. Methods for chemical defucosylation for example by using sulphuric acid are know so that major part of the oligosaccharide structures remain intact. The present invention is further preferably directed to remodelling of saccharides including oligosaccharide or glycopeptides from natural sources to structures according to the invention. The use of purified or isolated saccharide mictures for remodelling are preferred because the larger stability of the saccharides in comparision to protein materials. The present invention is specifically directed to remodelling of natural saccharides, preferably after desialylation and/or fucosylation, by α3-sialylation. For other purposes the present invention is furteher directed to remodelling of relased oligosaccharides by α3-galactosylation, α4-galactosylation, β4-GalNAc-transfer, β3-GalNAc-transfer, β3GlcNac-transfer, α6-sialylation, α3-fucosylation, α2-fucosylation, α6-fucosylation, more preferably α3-galactosylation, α4-galactosylation, β3GlcNAc-transfer or α6-sialylation, and in a preferred embodiment β3GlcNAc-transfer or α6-sialylation. The glycosylation reactions can be performed by glycosylatransferase enzymes, transglycosylating glycosidases, mutated transglycosylating enzymes described by S. whithers and colleagues by methods know in the art.

The present invention is directed to a method to remodel natural food material derived relased or isolated saccharides involving following steps
1) release of the saccharides chemically or enzymatically,
2) isolation of a crude oligosaccharides optionally enriched with desired saccharides which are preferably poly-N-acetyllactosamines,
3) release of terminal monosaccharide, preferably fucose and/or sialic acid, more preferably sialic acid, preferably the release is performed by mild acid treatment and
4) transferring a monosaccharide to the oligosaccharide to the saccharide mixture, preferably α3-linked sialic acid by a glycosyltransferase or transsialidase enzyme.

The present invention is directed to a method to remodel natural food material derived relased or isolated saccharides involving following steps
1) release of the saccharides chemically or enzymatically,
2) isolation of a crude oligosaccharides optionally enriched with desired saccharides which are preferably poly-N-acetyllactosamines,
3) optionally release of terminal monosaccharide, preferably fucose and/or sialic acid, more preferably sialic acid, and
4) transferring a monosaccharide to the oligosaccharide to the saccharide mixture, preferably α3-linked sialic acid by a α3-sialyltransferase or transsialidase enzyme.

In a preferred embodiment the methods further includes purification of oligosaccharides comprising different amounts of sialic acid, preferably fractions comprising one, two, three or four sialic acids or mixtures there of preferably ion exchange chromatograpy or by affinity chromatography.

In a preferedd embodiment the poly-Nacetylactosamine fraction is isolated by size exclusion chromatography or by affinity chromatography, preferably using a lectin binding poly-N-acetyllactosamines, preferably a poly-N-acetylactosamine binding lectin from food such as prefferred tomato lectin, potato lectin or wheat germ agglutinin.

Preferred poly-N-acetyllactosamine glycans contains at least one Galβ4Glcβ3Galβ4Glc(NAc)_{0 or 1} Galβ4Glcβ3Galβ structure as such or part of a larger glycan structures.

In a specific embodiment the present invention is directed to the methods above when the step 4) involves to two step method involving transfer Galβ4 and thereafter a terminal monosaccharide on that preferably α3-linked sialic acid by α3-sialyltransferase. In this methods raw materials containing terminal GlcNAc residues

Transsialidase enzymes have been obtained and cloned fro example from trypanosoma parasites.

Novel Carbon Modified Neutral or Acidic Sialic Acid Derivatives Having Binding Activities Towards H. pylori

The present invention further describes novel sialic acid derivatives with binding affinity towards Helicobacter pylori. The derivatives have the structure
SA(X—R)
wherein X is a linking atom or group bound to C1 of sialic acid and/or one or several of the following: C7, C8, C8 of sialic acid, the sialic acid may also be a sialic acid derivative lacking C8 or C8 and C9; especially, when R group is linked to the C7 or C8 of sialic acid, the sialic acid is preferably a truncated derivative of sialic acid lacking the structures including C7 and C8, or C9, respectively; R is H or an organic radical comprising more than 3 carbon atoms. Preferably X is —NH forming amide structure with the carboxylic acid group of the sialic acid residue. Preferably R is H or a C₄-C₃₀ organic radical comprising a ring structure and/or an aliphatic chain. More preferably R is a C₆-C₂₄ organic radical and most preferably R is a C6-24 aliphatic alkyl chain. Preferably the sialic acid is NeuNAc.

It was found out that the sialic acid can be modified to both carboxylic acid and/or glycerol-tail structures C7-C9. The modification of the glycerol tail after truncation of the structure preferably to C7 level with hydrophobic structures was found to be especially effective modification. In a preferred embodiment the modification group is a linear aliphatic alkyl chain. It is further realized that the modified sialic acid structure can be used together with other structures according to the invention. It is especially preferred to use C7-C9 modified structures with the polylactosamine type structures according to the invention. It is further preferred to use of the structures for screening and design other inhibitors for H. pylori.

The invention is further directed to analogs and derivatives of the modified sialic acid structure.

More preferably the sialic acid structure is linked to lactose or N-acetylactosamine structure. In a preferred embodiment the sialic acid derivative is α3-linked to type two N-acetyllactosamine sequence:
SA(X—R)αxGal{β4GlcNAc[β3Gal(β4Glc)ₚ₁]ₚ₂}ₚ₃
wherein X is linkage position of the sialic acid derivative as described above and integers p1, p2 and p3 are independently 0 or 1 indicating the presence or absence of the whole structure in { }, [ ] or ( ).

The term “oligosaccharide sequences” (OSs) include herein also modified sialic acid structures.

The present invention is also directed to the polyvalent conjugates of H. pylori binding oligosaccharide sequences or sialic acid modificated structures disclosed in the invention or analogs or derivatives thereof, the polyvalent conjugate being, for instance, according to the following structure: position C1 of reducing end terminal of the oligosaccharide sequence (OS) or R-group of modified sialic acid comprising the H. pylori binding terminal sequence of the invention is linked (-L-) to an oligovalent or a polyvalent carrier (Z), via a spacer group (Y) and optionally via a monosaccharide or oligosaccharide residue (X), forming a structure according to Formula
[OS—(X)ₙ-L-Y]ₘ-Z
where integer m has values m≧1, preferably m>1, and n is independently 0 or 1; L is oxygen, nitrogen, sulfur or carbon atom, X is preferably lactosyl-, galactosyl-, poly-N-acetyl-lactosaminyl, or part of an O-glycan or an N-glycan oligosaccharide sequence, Y is an aglycon spacer group or a linkage to Z; X comprises at least one mannose or N-acetylgalactosamine residue or Z comprises a carbohydrate material, such as a polysaccharide.

It is further realized that the novel sialic acid derivatives can be presented on poly-N-acetyllactosamines as described for NeuNAc according to the invention and on other types of glycoconjugates with binding activity towards H. pylori.

It is realized that the neutral sialic acid epitopes can bind different receptors than acidic NeuNAc on the surface of H. pylori. The invention specifically aims for use of the novel receptor for identification of corresponding adhesin from the surface of H. pylori. The present invention is further directed to design of potential new ligands inhibiting H. pylori binding to human and animal cells and tissues or for agglutination of the bacterium. The design and synthesis of the novel H. pylori binding substances is further directed to use of modeling with other carbohydrate structures binding the same receptor of the H. pylori.

In this invention the terms “analog” and “derivative” are defined as follows. According to the present invention it is possible to design structural analogs or derivatives of the Helicobacter pylori binding oligosaccharide sequences. Thus, the invention is also directed to the structural analogs of the substances according to the invention. The structural analogs according to the invention comprises the structural elements important for the binding of Helicobacter pylori to the oligosaccharide sequences. For design of effective structural analogs it is necessary to know the structural element important for the binding between Helicobacter pylori and the saccharides. The important structural elements are preferably not modified or these are modified by a very close mimetic of the important structural element.

The structural derivatives according to the invention are oligosaccharide sequences according to the invention modified chemically so that the binding to the Helicobacter pylori is retained or increased. According to the invention it is preferred to derivatize one or several of the hydroxyl or acetamido groups of the oligosaccharide sequences. The invention describes several positions of the molecules which could be changed when preparing the analogs or the derivatives. The hydroxyl or acetamido groups which preferably tolerate at least certain modifications are self-evident for a skilled artisan from the formulas described herein.

Bulky or acidic substituents and other structures, such as monosaccharide residues, are not tolerated, but methods to produce oligosaccharide analogs e.g. for the binding of a lectin are well known. For example, numerous analogs of sialyl-Lewis x oligosaccharide has been produced, representing the active functional groups different scaffold, see page 12090 Sears and Wong 1996. Similarily analogs of heparin oligosaccharides has been produced by Sanofi corporation and sialic acid mimicking inhibitors such as Zanamivir and Tamiflu (Relenza) for the sialidase enzyme by numerous groups. Preferably the oligosaccharide analog is build on a molecule comprising at least one six- or five-membered ring structure, more preferably the analog contains at least two ring structures comprising 6 or 5 atoms. In mimicking structures monosaccharide rings may be replaced rings such as cyclohexane or cyclopentane, aromatic rings including benzene ring, heterocyclic ring structures may comprise beside oxygen for example nitrogen and sulphur atoms. To lock the active ring conformations the ring structures may be interconnected by tolerated linker groups. Typical mimetic structure may also comprise peptide analog-structures for the oligosaccharide sequence or part of it.

The effects of the active groups to binding activity are cumulative and lack of one group could be compensated by adding an active residue on the other side of the molecule. Molecular modelling, preferably by a computer can be used to produce analog structures for the Helicobacter pylori binding oligosaccharide sequences according to the invention. The results from the molecular modelling of several oligosacharide sequences are given in examples and the same or similar methods, besides NMR and X-ray crystallography methods, can be used to obtain structures for other oligosaccharide sequences according to the invention. To find analogs the oligosaccharide structures can be “docked” to the carbohydrate binding molecule(s) of H. pylori, most probably to lectins of the bacterium and possible additional binding interactions can be searched.

It is also noted that the monovalent, oligovalent or polyvalent oligosaccharides can be activated to have higher activity towards the lectins by making derivative of the oligosaccharide by combinatorial chemistry. When the library is created by substituting one or few residues in the oligosacharide sequence, it can be considered as derivative library, alternatively when the library is created from the analogs of the oligosaccharide sequences described by the invention. A combinatorial chemistry library can be built on the oligosaccharide or its precursor or on glycoconjugates according to the invention. For example, oligosaccharides with variable reducing end can be produced by so called carbohydrid technology.

In a preferred embodiment a combinatorial chemistry library is conjugated to the Helicobacter pylori binding substances described by the invention. In a more preferred embodiment the library comprises at least 6 different molecules. Such library is preferred: use of assaying microbial binding to the oligosaccharide sequences according to the invention. A high affinity binder could be identified from the combinatorial library for example by using an inhibition assay, in which the library compounds are used to inhibit t bacterial binding to the glycolipids or glycoconjugates described by the invention. Structu analogs and derivatives preferred according to the invention can inhibit the binding of the Helicobacter pylori binding oligosaccharide sequences according to the invention to Helicobacter pylori.

In the following the Helicobacter pylori binding sequence is described as an oligosacchari sequence. The oligosaccharide sequence defined here can be a part of a natural or synthetic glycoconjugate or a free oligosaccharide or a part of a free oligosaccharide. Such oligosaccharide sequences can be bonded to various monosaccharides or oligosaccharides polysaccharides on polysaccharide chains, for example, if the saccharide sequence is expressed as part of a bacterial polysaccharide. Moreover, numerous natural modifications of monosaccharides are known as exemplified by O-acetyl or sulphated derivative of oligosaccharide sequences. The Helicobacter pylori binding substance defined here can comprise the oligosaccharide sequence described as a part of a natural or synthetic glycoconjugate or a corresponding free oligosaccharide or a part of a free oligosaccharide. The Helicobacter pylori binding substance can also comprise a mix of the Helicobacter pylori binding oligosaccharide sequences.

The Helicobacter pylori binding substances may be part of a saccharide chain or a glycoconjugate or a mixture of glycocompounds containing other known Helicobacter binding epitopes, with different saccharide sequences and conformations, such as Lewis b. Fucα2Galβ3(Fucα4)GlcNAc, or Neu5Acα3Galβ4Glc/GlcNAc. Using several binding substances together may be beneficial for therapy.

The present invention is specifically directed to the design of analogues of the oligosaccharide structures according to the invention comprising ring structures analogous to the monosaccharide residues of the oligosaccharide residues. More preferably the analogues are tested for binding or inhibition of Helicobacter pylori and best binding sequences are selected for development of a product. In another embodiment the molecules according to the invention or analogues or derivatives are tested for binding of other microbes or viruses, preferably for binding to toxin A of Clostridium difficile. Preferably the oligosaccharide analog is build on a molecule comprising at least one six- or five-membered ring structure, more preferably the analog contains at least two ring structures comprising 6 or 5 atoms. A preferred analogue type of the oligosaccharide comprise a terminal uronic acid amide or analogue or derivative thereof linked to Gal/GalNAcβ4GlcNAc-saccharide mimicking structure. According to the invention 2 and 4 hydroxyl groups of the terminal monosaccharide residue are not important for binding and 6-hydroxyl can be modified to structures actually increasing the affinity of the molecule, high affinty analogs can be produced when these positions are modified. The data shows that it is possible to design analogs which do not comprise all hydroxyl groups of the terminal monosaccharide residue. The present invention is specifically directed to attaching various organic derivatization molecule such as aromatic or aliphatic cyclic organic residue for the 3-position of terminal Gal/GalNAc production functional analog design. The derivatization may be produced by special linker chemistry allowing linking the cyclic organic residues to 3-position of Gal/GalNAcβ4. The geometry and even length of linking structures may be different from glycosidic bond structures provided that the cyclic organic residue can have at least some of the positive binding interactions of the corresponding terminal monosaccharide residue, especially close to the position of 6-hydroxyl/carboxyl/amide of the terminal monosaccharide residue.

In a specific embodiment the analogs are produced from amine group by replacement of hydroxyl group at position 3 of Gal/GalNAcβ4.

The present invention is specifically directed to the screening of analogs comprising terminal cyclic molecule on terminal 3-position of Gal/GalNAc in the trisaccharide epitope for binding to Helicobacter pylori. The terminal cyclic molecule is preferably a six membered organic residue, and more preferably it also comprises a carboxylic acid, an amide or alkyl amide structure similar to the structures terminal and/or in the middle hexuronic acids in the formulas according the invention.

Alternatively terminal uronic acid amide or analogue or derivative is 1-3-linked to Gal, which is linked to the GlcNAc mimicking structure. In mimicking structures monosaccharide rings may be replaced by rings such as cyclohexane or cyclopentane, aromatic rings including benzene ring, heterocyclic ring structures may comprise beside oxygen for example nitrogen and sulphur atoms. To lock the active ring conformations the ring structures may be interconnected by tolerated linker groups. Typical mimetic structure may also comprise peptide analog-structures for the oligosaccharide sequence or part of it. The present invention is also directed to the design and/or screening of peptide analogs for the oligosaccharide sequences. Furthermore the present invention is directed for screening of DNA or RNA-based analogues, for example so called aptamers, of the oligosaccharide sequences according to the invention. The effects of the active groups to binding activity are cumulative and lack of one group could be compensated by adding an active residue on the other side of the molecule.

Molecular modelling, preferably by a computer, can be used to produce analog structures for the Helicobacter pylori binding oligosaccharide sequences according to the invention. The results from the molecular modelling of several oligosacharide sequences are given in examples and the same or similar methods, besides NMR and X-ray crystallography methods, can be used to obtain structures for other binding oligosaccharide sequences or analogues or derivatives according to the invention. In a preferred embodiment the analogues or derivatives of the oligosaccharide structures having same or similar conformations with the oligosaccharide structures according to the invention are selected from computer assisted molecular modeling results or screening database or databases containing three dimensional structures of molecules, these methods are referred as computerized screening methods.

The present invention is further directed to the testing oligosaccharide structures and analogues and derivatives thereof which are selected by the computerized screening methods for binding to other pathogenic microbes or viruses or toxins having a binding specificity similar to the binding specificity of Helicobacter pylori with regard to one or several oligosaccharide sequences according to the invention. In a preferred embodiment the oligosaccharide structures and analogues and derivatives selected by the computerized screening methods are tested for binding to toxin A of Clostridium difficile.

The analogue molecules can be synthetically produced or obtained from natural sources. Molecules can also be produced virtually in computers and part of the screening of the active molecules can also be performed in silico. The present invention is also directed to the searching of Helicobacter pylori-binding and/or inhibiting analogues and/or derivatives for the oligosaccharide structures according to the invention by computerized fitting of a carbohydrate structure, analogue or derivative to a carbohydrate binding site on H. pylori.

The Helicobacter pylori-binding oligosacchride sequence, analogues or derivatives thereof are “docked” by methods of molecular modeling to the carbohydrate binding molecule(s) of Helicobacter pylori, most probably to lectins of the bacterium and additional binding interactions are searched. The computerized docking of a three dimensional structure of the oligosaccharide sequence on a three dimensional model of a carbohydrate binding site further helps the design of binding active analogues by allowing determination of binding interactions and positions for possible additional binding interactions. The method is also directed to the comparison of the binding of the oligosaccharide structures and analogues and derivatives thereof by the computerized docking methods.

The present invention is further directed to the testing of oligosaccharide structures and analogues and derivatives thereof by the computerized docking methods for binding to other pathogenic microbes or viruses or toxins having a binding specificity similar to the binding specificity of Helicobacter pylori with regard to one or several oligosaccharide sequences according to the invention. In a preferred embodiment the oligosaccharide structures and analogues and derivatives thereof are tested for binding to toxin A of Clostridium difficile by the computerized docking methods.

It is also noted that the monovalent, oligovalent or polyvalent oligosaccharides can be activated to have higher activity towards lectins by making a derivative of the oligosaccharide by combinatorial chemistry. When a library is created by substituting one or few residues in the oligosaccharide sequence, it can be considered as a derivative library. Alternatively, when the library is created from the analogs of the oligosaccharide sequences described by the invention, it can be considered as an analog library. A combinatorial chemistry library can be built on the oligosaccharide or its precursor or on glycoconjugates according to the invention. For example, oligosaccharides with variable reducing end can be produced by so called carbohydrid technology. The present invention is directed to the design and production of a combinatorial chemistry library, a multide of chemical analogues and/or derivatives of the oligosaccharide structures according to the invention, and testing these for binding or inhibition of Helicobacter pylori. The present invention is further directed to the testing of the combinatorial chemistry library for binding to other pathogenic microbes or viruses or toxins having a binding specificity similar to the binding specificity of Helicobacter pylori with regard to one or several oligosaccharide sequences according to the invention. In a preferred embodiment the combinatorial chemistry library is tested for binding to toxin A of Clostridium difficile.

In a preferred embodiment a combinatorial chemistry library is conjugated to the Helicobacter pylori binding substances described by the invention. In a more preferred embodiment the library comprises at least 6 different molecules. Preferably the combinatorial chemistry modifications are produced by different amides from carboxylic acid group on R₈ or R₉ according to Formula 9. Group to be modified in R₈ may also be an aldehyde or amine or another type of reactive group. Such library is preferred for use of assaying microbial binding to the oligosaccharide sequences according to the invention. Amino acids or collections of organic amides are commercially available, which substances can be used for the synthesis of combinatorial library of uronic acid amides. A high affinity binder could be identified from the combinatorial library for example by using an inhibition assay, in which the library compounds are used to inhibit the bacterial binding to the glycolipids or glycoconjugates described by the invention. Structural analogs and derivatives preferred according to the invention can inhibit the binding of the Helicobacter pylori binding oligosaccharide sequences according to the invention to Helicobacter pylori.

In the following the Helicobacter pylori binding sequence is described as an oligosaccharide sequence. The oligosaccharide sequence defined here can be a part of a natural or synthetic glycoconjugate or a free oligosaccharide or a part of a free oligosaccharide. Such oligosaccharide sequences can be bonded to various monosaccharides or oligosaccharides or polysaccharides on polysaccharide chains, for example, the saccharide sequence is expressed as part of a bacterial polysaccharide. Moreover, numerous natural modifications of monosaccharides are known as exemplified by O-acetyl or sulphated derivatives of the oligosaccharide sequences. In a limited embodiment the oligosaccharide sequence means terminal non-reducing end oligosaccharide sequence which is not modified by any other monosaccharide residue, except optionally at the reducing end. Preferably in broadest sense the term oligosaccharide sequence includes structural analogues and derivatives of the oligosaccharide structures according to the invention, preferably as described by the invention, having same or similar binding activity with regard to the H. pylori. The Helicobacter pylori binding substance defined here can comprise the oligosaccharide sequence described as a natural or synthetic glycoconjugate or part thereof or a corresponding free oligosaccharide or a part of a free oligosaccharide. The Helicobacter pylori binding substance can also comprise a mixture of the Helicobacter pylori binding oligosaccharide sequences.

The Helicobacter pylori binding oligosaccharide sequences can be synthesized enzymatically by glycosyltransferases, or by transglycosylation catalyzed by glycosidase or transglycosidase enzymes (Ernst et al., 2000). Specifities of these enzymes and the use of co-factors can be engineered. Specific modified enzymes can be used to obtain more effective synthesis, for example, glycosynthase is modified to do transglycosylation only. Organic synthesis of the saccharides and the conjugates described herein or compounds similar to these are known (Ernst et al., 2000). Saccharide materials can be isolated from natural sources and modified chemically or enzymatically into the Helicobacter pylori binding compounds. Natural oligosaccharides can be isolated from milks produced by various ruminants.

Transgenic organisms, such as cows or microbes, expressing glycosylating enzymes can be used for the production of saccharides.

The bacterium binding substances are preferably represented in clustered form such as by glycolipids on cell membranes, micelles, liposomes, or on solid phases such as TCL-plates used in the assays. The clustered representation with correct spacing creates high affinity binding.

According to the invention it is also possible to use the Helicobacter pylori binding epitopes or naturally occurring, or a synthetically produced analogue or derivative thereof having a similar or better binding activity with regard to Helicobacter pylori. It is also possible to use a substance containing the bacterium binding substance such as a receptor active ganglioside described in the invention or an analogue or derivative thereof having a similar or better binding activity with regard to Helicobacter pylori. The bacterium binding substance may be a glycosidically linked terminal epitope of an oligosaccharide chain. Alternatively the bacterium binding epitope may be a branch of an oligosaccharide chain, preferably a polylactosamine chain.

The Helicobacter pylori binding substance may be conjugated to an antibiotic substance, preferably a penicillin type antibiotic. The Helicobacter pylori binding substance targets the antibiotic to Helicobacter pylori. Such conjugate is beneficial in treatment because a lower amount of antibiotic is needed for treatment or therapy against Helicobacter pylori, which leads to lower side effect of the antibiotic. The antibiotic part of the conjugate is aimed at killing or weaken the bacteria, but the conjugate may also have an antiadhesive effect as described below.

The bacterium binding substances, preferably in oligovalent or clustered form, can be used to treat a disease or condition caused by the presence of the Helicobacter pylori. This is done by using the Helicobacter pylori binding substances for anti-adhesion, i.e. to inhibit the binding of Helicobacter pylori to the receptor epitopes of the target cells or tissues. When the Helicobacter pylori binding substance or pharmaceutical composition is administered it will compete with receptor glycoconjugates on the target cells for the binding of the bacteria. Some or all of the bacteria will then be bound to the Helicobacter pylori binding substance instead of the receptor on the target cells or tissues. The bacteria bound to the Helicobacter pylori binding substances are then removed from the patient (for example by the fluid flow in the gastrointestinal tract), resulting in reduced effects of the bacteria on the health of the patient. Preferably the substance used is a soluble composition comprising the Helicobacter pylori binding substances. The substance can be attached to a carrier substance which is preferably not a protein. When using a carrier molecule several molecules of the Helicobacter pylori binding substance can be attached to one carrier and inhibitory efficiency is improved.

The target cells are primarily epithelial cells of the target tissue, especially the gastrointestinal tract, other potential target tissues are for example liver and pancreas. Glycosylation of the target tissue may change because of infection by a pathogen (Karlsson et al., 2000). Target cells may also be malignant, transformed or cancer/tumour cells in the target tissue. Transformed cells and tissues express altered types of glycosylation and may provide receptors to bacteria. Binding of lectins or saccharides (carbohydrate-carbohydrate interaction) to saccharides on glycoprotein or glycolipid receptors can activate cells, in case of cancer/malignant cells this may be lead to growth or metastasis of the cancer. Several of the oligosaccharide epitopes and sialylated polylactosamines from malignant cells (Stroud et al., 1996), have been reported to be cancer-associated or cancer antigens. Helicobacter pylori is associated with gastric lymphoma. The substances described herein can be used to prevent binding of Helicobacter pylori to premalignant or malignant cells and activation of cancer development or metastasis. Inhibition of the binding may cure gastric cancer, especially lymphoma.

Target cells also includes blood cells, especially leukocytes. It is known that Helicobacter pylori strains associated with peptic ulcer, as the strain mainly used here, stimulates an inflammatory response from granulocytes, even when the bacteria are nonopsonized (Rautelin et al., 1994a,b). The initial event in the phagocytosis of the bacterium most likely involves specific lectin-like interactions resulting in the agglutination of the granulocytes (Ofek and Sharon, 1988). Subsequent to the phagocytotic event oxidative burst reactions occur which may be of consequence for the pathogenesis of Helicobacter pylori-associated diseases (Babior, 1978). Several sialylated and non-acid glycosphingolipids having repeating N-acetyllactosamine units have been isolated and characterized from granulocytes (Fukuda et al., 1985; Stroud et al., 1996) and may thus act as potential receptors for Helicobacter pylori on the white blood cell surface. Furthermore, also the X₂ glycosphingolipid has been isolated from the same source (Teneberg, S., unpublished). The present invention confirms the presence of receptor saccharides on human erythrocytes and granulocytes which can be recognized by an N-acetyllactosamine specific lectin and by a monoclonal antibody (X₂, GalNAcβ3Galβ4GlcNAc-). The Helicobacter pylori binding substances can be useful to inhibit the binding of leukocytes to Helicobacter pylori and in prevention of the oxidative burst and/or inflammation following the activation of leukocytes.

It is known that Helicobacter pylori can bind several kinds of oligosaccharide sequences. Some of the binding by specific strains may represent more symbiotic interactions which do not lead to cancer or severe conditions. The present data about binding to cancer-type saccharide epitopes indicates that the Helicobacter pylori binding substance can prevent more pathologic interactions, in doing this it may leave some of the less pathogenic Helicobacter pylori bacteria/strains binding to other receptor structures. Therefore total removal of the bacteria may not be necessary for the prevention of the diseases related to Helicobacter pylori. The less pathogenic bacteria may even have a probiotic effect in the prevention of more pathogenic strains of Helicobacter pylori.

It is also realized that Helicobacter pylori contains large polylactosamine oligosaccharides on its surface which at least in some strains contains non-fucosylated epitopes which can be bound by the bacterium as described by the invention. The substance described herein can also prevent the binding between Helicobacter pylori bacteria and that way inhibit bacteria for example in process of colonization.

According to the invention it is possible to incorporate the Helicobacter pylori binding substance, optionally with a carrier, in a pharmaceutical composition, which is suitable for the treatment of a condition due to the presence of Helicobacter pylori in a patient or to use the Helicobacter pylori binding substance in a method for treatment of such conditions. Examples of conditions treatable according to the invention are chronic superficial gastritis, gastric ulcer, duodenal ulcer, non-Hodgkin lymphoma in human stomach, gastric adenocarcinoma, and certain pancreatic, skin, liver, or heart diseases, sudden infant death syndrome, autoimmune diseases including autoimmune gastritis and pernicious anaemia and non-steroid anti-inflammatory drug (NSAID) related gastric disease, all, at least partially, caused by the Helicobacter pylori infection.

The pharmaceutical composition containing the Helicobacter pylori binding substance may also comprise other substances, such as an inert vehicle, or pharmaceutically acceptable carriers, preservatives etc, which are well known to persons skilled in the art. The Helicobacter pylori binding substance can be administered together with other drugs such as antibiotics used against Helicobacter pylori.

The Helicobacter pylori binding substance or pharmaceutical composition containing such substance may be administered in any suitable way, although an oral administration is preferred.

The term “treatment” used herein relates both to treatment in order to cure or alleviate a disease or a condition, and to treatment in order to prevent the development of a disease or a condition. The treatment may be either performed in a acute or in a chronic way.

The term “patient”, as used herein, relates to any human or non-human mammal in need of treatment according to the invention.

It is also possible to use the Helicobacter pylori binding substance to identify one or more adhesins by screening for proteins or carbohydrates (by carbohydrate-carbohydrate interactions) that bind to the Helicobacter pylori binding substance. The carbohydrate binding protein may be a lectin or a carbohydrate binding enzyme. The screening can be done for example by affinity chromatography or affinity cross linking methods (Ilver et al., 1998).

Furthermore, it is possible to use substances specifically binding or inactivating the Helicobacter pylori binding substances present on human tissues and thus prevent the binding of Helicobacter pylori. Examples of such substances include plant lectins such as Erythrina cristagalli and Erythrina corallodendron lectins (Teneberg et al., 1994) or polylactosamine binding lectins such as potatolectin or NeuNAcα3 specific lectins such as Sambucus nigra agglutinin. When used in humans, the binding substance should be suitable for such use such as a humanized antibody or a recombinant glycosidase of human origin which is non-immunogenic and capable of cleaving the terminal monosaccharide residue/residues from the Helicobacter pylori binding substances. However, in the gastrointestinal tract, many naturally occuring lectins and glycosidases originating for example from food are tolerated.

Furthermore, it is possible to use the Helicobacter pylori binding substance as part of a nutritional composition including food- and feedstuff. It is preferred to use the Helicobacter pylori binding substance as a part of so called functional or functionalized food. The said functional food has a positive effect on the person's or animal's health by inhibiting or preventing the binding of Helicobacter pylori to target cells or tissues. The Helicobacter pylori binding substance can be a part of a defined food or functional food composition. The functional food can contain other acceptable food ingredients accepted by authorities such as Food and Drug Administration in the USA. The Helicobacter pylori binding substance can also be used as a nutritional additive, preferably as a food or a beverage additive to produce a functional food or a functional beverage. The food or food additive can also be produced by having, e.g., a domestic animal such as a cow or other animal produce the Helicobacter pylori binding substance in larger amounts naturally in its milk. This can be accomplished by having the animal overexpress suitable glycosyltransferases in its milk. A specific strain or species of a domestic animal can be chosen and bred for larger production of the Helicobacter pylori binding substance. The Helicobacter pylori binding substance for a nutritional composition or nutritional additive can also be produced by a micro-organisms such as a bacteria or a yeast.

It is especially useful to have the Helicobacter pylori binding substance as part of a food for an infant, preferably as a part of an infant formula. Many infants are fed by special formulas in replacement of natural human milk. Helicobacter pylori is especially infective with regard to infants or young children, and considering the diseases it may later cause it is reasonable to prevent the infection. Helicobacter pylori is also known to cause sudden infant death syndrome, but the strong antiobiotic treatments used to eradicate the bacterium may be especially unsuitable for young children or infants.

Furthermore, it is possible to use the Helicobacter pylori binding substance in the diagnosis of a condition caused by an Helicobacter pylori infection. Diagnostic uses also include the use of the Helicobacter pylori binding substance for typing of Helicobacter pylori. When the substance is used for diagnosis or typing, it may be included in, e.g., a probe or a test stick, optionally constituting a part of a test kit. When this probe or test stick is brought into contact with a sample containing Helicobacter pylori, the bacteria will bind the probe or test stick and can be thus removed from the sample and further analyzed.

Terminal residues include preferably β3-linked glucuronic acid and more preferably 6-amides such as methylamide thereof. Therefore analogs and derivatives of the sequence can be produced by changing or derivatising the terminal 6-position of the trisaccharide epitopes.

Preferred Helicobacter pylori Binding Substances

The oligosaccharide sequences according to the invention were found to be unexpectedly effective binders when presented on thin layer surface. This method allows polyvalent presentation of the glycolipid sequences. The surprisingly high activity of the polyvalent presentation of the oligosaccharide sequences makes polyvalency a preferred way to represent the oligosaccharide sequences of the invention.

The glycolipid structures are naturally presented in a polyvalent form on cellular membranes. This type of representation can be mimicked by the solid phase assay described below or by making liposomes of glycolipids or neoglycolipids.

The present novel neoglycolipids produced by reductive amination of hydrophobic hexadecylaniline were able to provide effective presentation of the oligosaccharides. Most previously known neoglycolipid conjugates used for binding of bacteria have contained a negatively charged groups such as phosphor ester of phosphadityl ethanolamine neoglycolipids. Problems of such compounds are negative charge of the substance and natural biological binding involving the phospholipid structure. Negatively charged molecules are known to be involved in numerous non-specific bindings with proteins and other biological substances. Moreover, many of these structures are labile and can be enzymatically or chemically degraded. The present invention is directed to the non-acidic conjugates of oligosaccharide sequences meaning that the oligosaccharide sequences are linked to non-acidic chemical structures. Preferably, the non-acidic conjugates are neutral meaning that the oligosaccharide sequences are linked to neutral, non-charged, chemical structures. The preferred conjugates according to the invention are polyvalent substances.

In the previous art bioactive oligosaccharide sequences are often linked to carrier structures by reducing a part of the receptor active oligosaccharide structure. Hydrophobic spacers containing alkyl chains (—CH₂—)ₙ and/or benzyl rings have been used. However, hydrophobic structures are in general known to be involved in non-specific interactions with proteins and other bioactive molecules.

The neoglycolipid data of the examples below show that under the experimental conditions used in the assay the hexadecylaniline parts of the neoglycolipid compounds do not cause non-specific binding for the studied bacterium. In the neoglycolipids the hexadecylaniline part of the conjugate forms probably a lipid layer like structure and is not available for the binding. The invention shows that reducing a monosaccharide residue belonging to the binding epitope may destroy the binding. It was further realized that a reduced monosaccharide can be used as a hydrophilic spacer to link a receptor epitope and a polyvalent presentation structure. According to the invention it is preferred to link the bioactive oligosaccharide via a hydrophilic spacer to a polyvalent or multivalent carrier molecule to form a polyvalent or oligovalent/multivalent structure. All polyvalent (comprising more than 10 oligosaccharide residues) and oligovalent/multivalent structures (comprising 2-10 oligosaccharide residues) are referred here as polyvalent structures, though depending on the application oligovalent/multivalent constructs can be more preferred than larger polyvalent structures. The hydrophilic spacer group comprises preferably at least one hydroxyl group. More preferably the spacer comprises at least two hydroxyl groups and most preferably the spacer comprises at least three hydroxyl groups.

According to the invention the hydrophilic spacer group is preferably a flexible chain comprising one or several —CHOH— groups and/or an amide side chain such as an acetamido —NHCOCH₃ or an alkylamido. The hydroxyl groups and/or the acetamido group also protects the spacer from enzymatic hydrolysis in vivo. The term flexible means that the spacer comprises flexible bonds and do not form a ring structure without flexibility. A reduced monosaccharide residues such as ones formed by reductive amination in the present invention are examples of flexible hydrophilic spacers. The flexible hydrophilic spacer is optimal for avoiding non-specific binding of neoglycolipid or polyvalent conjugates. This is essential optimal activity in bioassays and for bioactivity of pharmaceuticals or functional foods, for example.

A general formula for a conjugate with a flexible hydrophilic linker has the following Formula IV:
[OS—O—(X)ₙ-L₁-CH(H/{CH₁₋₂OH}ₚ₁)—{CH₁OH}ₚ₂—{CH(NH—R)}ₚ₃—{CH₁OH}ₚ₄-L₂]ₘ-Z
wherein L₁ and L₂ are linking groups comprising independently oxygen, nitrogen, sulphur or carbon linkage atom or two linking atoms of the group forming linkages such as —O—, —S—, —CH₂—, —N—, —N(COCH3)-, amide groups —CO—NH— or —NH—CO— or —N—N— (hydrazine derivative) or amino oxy-linkages —O—N— and —N—O—. L1 is linkage from carbon 1 of the reducing end monosaccharide of X or when n=0, L1 replaces —O— and links directly from the reducing end C1 of OS.

p1, p2, p3, and p4 are independently integers from 0-7, with the proviso that at least one of p1, p2, p3, and p4 is at least 1. CH₁₋₂OH in the branching term {CH₁₋₂OH}ₚ₁ means that the chain terminating group is CH₂OH and when the p1 is more than 1 there is secondary alcohol groups —CHOH— linking the terminating group to the rest of the spacer. R is preferably acetyl group (—COCH₃) or R is an alternative linkage to Z and then L₂ is one or two atom chain terminating group, in another embodiment R is an analog forming group comprising C₁₋₄ acyl group (preferably hydrophilic such as hydroxy alkyl) comprising amido structure or H or C₁₋₄ alkyl forming an amine. And m>1 and Z is polyvalent carrier. OS is Helicobacter pylori binding oligosaccharide sequence and X is a saccharide residue, which can be replaced by carbon (—C—), nitrogen (—N—) or sulphur (—S—) linkage.

Preferred polyvalent structures comprising a flexible hydrophilic spacer according to formula IV include Helicobacter pylori binding oligosaccharide sequence(OS) β1-3 linked to Galβ4Glc(red)-Z, and structures OSOβ6GlcNAc(red)-Z and OSOβ6GalNAc(red)-Z, where “(red)” means the amine linkage structure formed by reductive amination from the reducing end monosaccharides and an amine group of the polyvalent carrier Z.

In the present invention the oligosaccharide group is preferably linked in a polyvalent or an oligovalent form to a carrier which is not a protein or peptide to avoid antigenicity and possible allergic reactions, preferably the backbone is a natural non-antigenic polysaccharide.

Ex Vivo Uses of the Present Invention

It is realized that the present invention can be used for inhibition of pathogens especially diarrhea causing E. coli ex vivo and such method have use in disinfection and preservevation type applications. It is preferred to use the receptor oligosaccharide sequences according to the present invention as part of single substances or as single substances or more preferably as composition comprising at least two receptor oligosaccharide sequences from different groups according to the present invention for inhibition pathogens, preferably E. coli ex vivo. Polyvalent conjugates according to the present invention especially soluble polyvalent conjugates which can agglutinate pathogens, preferably diarrheagenic E. coli, are preferred for ex vivo uses. One special ex vivo embodiment of the invention is the cleansing or disinfection of surfaces, e.g., of tables, medical devices and packages, in hospital or hospital-like enviroment with a cleanser or disinfectant containing the receptor oligosaccharide sequences described in the present invention. The receptor saccharides described by the invention can also be used as ingredients in a soap or detergent used for washing or bathing of patients in hospital or hospital-like enviroment.

Oral Infections and Oral Health Products

It is realized that infections targetted by the present invention spread through oral route, possibly also from nose to the oral cavity. The present invention is directed to the prevention of the infections already in human mouth. The present invention is directed to the treatment of oral infections by at least two different oligosaccharide sequences which can inhibit at least two different binding specificities of pathogen, preferably orally infecting bacterium and more preferably a diarrhea causing bacterium. It is preferred to use the receptor oligosaccharide sequences according to the present invention as part of single substances or as single substances or as composition comprising at least two receptor oligosaccharide sequences from different groups according to the present invention for inhibition of oral or nasal infections. According to the present invention the receptor oligosaccharide sequences according to the present invention are used as compositions or as separate substances in products inhibiting pathogens, called here mouth hygiene products, in human mouth.

It is realized that human mouth comprises similar receptors as human intestine especially on proteins at least neolacto-receptors, mannose receptors and oligosaccharide receptors resembling fucose receptors according to the present invention. As a separate embodiment it is realized that the substances and compositions according to the present invention are also useful in inhibiting pathogens causing caries. In a specific embodiment the present invention is also directed to the compositions according to the present invention for treatment of other orally spreading infections such as infection causing otitis media or lung infections including influenza, bronchitis or pneumonia. The mouth hygiene products according to the present invention can also be directed against caries, otitis media, bronchitis and pneumonia. In a specific embodiment the composition to used in mouth hygiene product or for inhibition of a pathogen infecting orally comprises at least oligosaccharide sequences Neu5Acα3Galβ4GlcNAc and/or Neu5Acα3Galβ4Glc or more preferably Neu5Acα6Galβ4GlcNAc and/or Neu5Acα6Galβ4Glc and it is directed at least against human influenza virus, preferably for prophylaxis of influenza virus.

The present invention is especially directed to mouth hygiene products comprising substances or compositions comprising pathogen inhibiting oligosaccharide sequences, especially oligosaccharide sequences according to the invention. The mouth hygiene product is preferably selected from the group consisting of tooth pastes, mouth wash solutions, mouth tablets, chewing tablets, and chewing gums. It is preferred to use either monovalent receptor oligosaccharide sequences or polyvalent receptor oligosaccharide sequences. In another preferred embodiment the mouth hygiene product comprises polyvalent oligosaccharide sequences according to the present invention. Due to size of human mouth and volume of liquid saliva on its surface relatively small amount of oligosaccharides is enough to obtain saturating rating concentrations of pathogen inhibiting receptors in mouth. The typical amounts of receptor active monovalent epitopes varies from about 100 nmol to 100 μmol of the receptor active oligosaccharide, (at molecular weight 1000 Da this would be 100 μg to 100 mg). More generally useful amounts are estimated to be between about 1-10 μmol. In a separate embodiment the present invention about therapheutical composition is also directed to pathogen inhibiting nasal sprays. The nasal sprays can be directed against otitis media or lung infections.

Topical, Washing and Cosmetic Products

It is realized that the common pathogens can spread on human surfaces such as human skin, genital epithelia, hair, household surfaces, and other surfaces in human environment. The oligosacchride sequences according to the present invention are also useful for prevention of the pathogens also in these environments: It is therefore also preferred to use the oligosaccharide sequences according to the present invention as single substances, as part of single substances, or as composition comprising at least two receptor oligosaccharide sequences from different groups according to the present invention in topical or cosmetic products, for example as creams, lotions, or gels. It is also preferred to use the substances or compositions according to the present invention products aimed for washing human skin, hair or genital epithelia, (which can be also called as personal hygiene products), or for household surfaces, dishes or clothes. Traditional antibiotics have been aimed for use of household washing solutions, but are not useful because of resistance problems which are not likely with the substances according to the present invention. In preferred embodiment polyvalent oligosaccharide sequences are used for washing solutions, in another preferred embodiment monovalent oligosaccharide sequences are used for washing solutions.

Food Safety Products to be Applied to Foods or Feeds, Beverages, Drinks and Water

Besides the therapheutic uses in humans or in animals the invention is also directed to the use of receptors and compositions according to the invention for the prevention of the infections by using the invention to neutralize pathogens or bacteria inside or on surfaces of food products. Carbohydrates according to the present invention can for example be applied on the surfaces of meat products or animal bodies, body parts in meat production to prevent the spreading of pathogens. Use of soluble and other polyvalent conjugates to cover and agglutinate the bacteria are preferred. A specific method to be used on a surface of a solid or semi-solid food product involves contacting the bacteria with the carbohydrates receptors described by the invention and optionally washing away the pathogen carbohydrate complexes. This kind of method is not acceptable with traditional antibiotics. The carbohydrates according to the invention can be also applied to liquid food products or concentrates or powder to make these including milk and liquid milk like products, various beverages including juices, soft drinks, sport drinks, alcoholic beverages and the like.

In a specific embodiment the carbohydrate according to the invention in polymeric form is applied to a liquid food product or a beverage product, potential pathogens are agglutinated by the polyvalent conjugate and the agglutinated complex is removed by a method based on size or solubility of the complex. Non-soluble agglutinates can be removed when these precipitate by standard methods like decanting the solution above the precipitate or more usually more effectively by filtration methods. Filtration methods can be used to remove larger agglutinated complexes.

Preferred foods to be treated with carbohydrates according to the invention includes various animal food products, especially meat products and middle products in processing. Many pathogens including diarrhea causing E. coli bacteria are transmitted effectively from vegetables, fruits, salads and other plant foods which are not properly washed. The food stuffs needing washing, but not washed properly or washed with contaminated water are especially problematic in developing countries. The present invention is also directed to methods for increasing food safety of plant foods and other foods in need of washing to control the amount of pathogens, especially pathogenic E. coli bacteria in the food products. The invention is especially directed to home customer products and products aimed for the food industry to prevent infections from food. The product is preferentially in solid form as powder or pill or in a capsule containing solutions of the receptors according to the invention, which can be applied to food under processing. Such product can be used to prevent diarrheas in developing countries especially diarrheas in children. The food safety product is also directed to the prevention of travellers diarrheas. The food safety products and feed safety products below can be considered as novel safe preservatives.

Filter Products to Purify Beverages and Water

Contaminated drinks and water are major cause of gastrointestinal diseases, especially diarrheas. The receptors according to the present invention can be also used to make filters to purify pathogens, especially bacteria from liquid food and beverages and water, especially water used for drinking and preparing foods. Preferentially at least two recptor structures are used. Methods are known to produce solid phase materials to which carbohydrate sequences are conjugtated to be used as filters for example from cellulose or plastics or agarose and similar materials. The filters according to the invention also includes affinity chromatography material known in the art. Methods to remove bound materials from such filters are known and in a specific embodiment the filter is regenerated by removing the contaminant and optionally sterilizing the filter by heat or other sterilizing means.

Feed Safety Products

The food safety products described above can be also applied to animal solid and liquid feeds and drinking water of animals. Preferred target animals to be protected includes pet animals, especially cats and dogs and cattle or farm animal such as cows and other domestic ruminants, pigs, sheep, horses, poultry including for example hens, ducks and turkeys, and rabbits.

Water, Food and Feed Safety Analytics

Standard analytic and diagnostic methods in combination with the receptor carbohydrates according to the invention can be applied to water, beverages, foods and feeds to measure presence pathogens binding to the receptor carbohydrates. The knowledge of the binding specificities of contaminating pathogens can be applied to design of theraphy when patients are infected or to methods for food safety remove or control pathogens as described above.

Other Carbohydrate Based Interactions which can be Inhibited According to the Invention

Beside inhibiting different types of adhesin presentations the invention can be also used to inhibit carbohydrate-carbohydrate interactions and carbohydrate-lectin interactions.

The carbohydrate compositions and substances comprises of oligosaccharide sequences. The oligosaccharides inhibit one or several pathogens by binding one or several pathogens and/or by binding the receptors of one or several pathogens. Preferentially at least two pathogen inhibiting oligosaccharide sequences are used and more preferentially at least three pathogen inhibiting oligosaccharide sequences. In other embodiments at least four, five, six, or seven pathogenesis inhibiting oligosaccharide sequences are used.

In specific theraphies one or several of the oligosaccharide sequences are given separately at different time points. This is especially useful when the administration of all the oligosaccharide sequences would have negative effects on the normal flora. The separate administration of the therapheutic compositions can be useful also because of effect of nutritional situation in the gastrointestinal tract could change differently the stability of the on the oligosaccharide sequences according to the invention in the gastrointestinal tract.

Use of the Invention Together with Probiotic Bacteria

When the invention is used to inhibit bacterial binding, especially multiple bacterial bindings, also some beneficial bacterial bindings can be prevented. The normal bacterial flora has many important functions for example in the human gastrointestinal system. The destruction of the normal bacterial flora is however an even larger problem with use of traditional antibiotics.

In a separate embodiment at least two pathogen inhibiting oligosaccharides are administered together with a probiotic microbe and/or a prebiotic substance. The probiotic microbe according to the invention represent a non-harmful bacteria with beneficial functions, for example in digestion of food, providing nutrients and vitamins or covering tissue surfaces from pathogenic bacteria. The probiotic bacteria comprise preferentially one or several or multitude of normal bacterial flora. In a preferred embodiment the probiotic bacterium comprise one or several types, strains, or species of lactic acid bacteria.

The prebiotic substance is a substance supporting the normal flora or probiotic microbe. Preferred prebiotic substances include prebiotic carbohydrates, such as galactose oligosaccharides, xylose oligosaccharide, or fructose oligosaccharides used as prebiotic substances, the prebiotic substances also include polysaccharides and fibers with prebiotic acticities such as inulin or midified starches. The present invention is also directed to the use of other polysaccharides which are used in food or for nutritional purposes such as chitosan or beta-glucans for example glucan from oats, which are used to reduce cholesterol and fats. In a preferred embodiment one or several pathogen inhibiting carbohydrates are chosen so that they are also prebiotic substances like carbohydrates with a non-reducing terminal beta linked galactose residue In a preferred form of theraphy
a) pathogens and potentially part of the normal flora are first removed by one or more preferentially at least two carbohydrates according to the invention
b) probiotic microbe and/or prebiotic substance are applied.

Steps 1 and 2 may also be applied in reversed order, preferably with a large amount of the probiotic microbe and/or prebiotic substance and then step one. According to the invention it is also possible to repeat steps 1 and/or 2 several times while varying the order of the steps. Steps 1 and 2 may be applied at the same time. The substances according to the invention can be administered together with probiotic microbe and/or prebiotic substance or alternatively probiotic microbe and/or prebiotic substance can be included in the compositions according to the invention, and then steps 1 and 2 above can be performed simultaneously.

Some of the oligosaccharide sequences according to the invention are known to have prebiotic effects, these includes N-acetyl-lactosamine type oligosaccharide sequences, and fucosylated oligosaccharides, especially human milk oligosaccharides. Administration human milk oligosaccharides together with a probiotic microbe and/or prebiotic substance, especially N-acetyllactosamine containing for example one or several from the group Lacto-N-neotetraose, Lacto-N-tetraose, Lacto-N-hexaose, Lacto-N-neohexaose, para-Lacto-N-hexaose, para-Lacto-N-neohexaose, and/or fucosylated oligosaccharides derived from these such as and/or mono-di- or trifucosylated Lacto-N-tetraose (LNT) or/or Lacto-N-neotetraose (LNnT) and/or fucosyl-lactose oligosaccharides such as 2′fucosyl-lactose, and/or 3-fucosyllactose, and/or difucosyllactose.

Other Useful Substances to be Used with the Substances and/or Compositions According to the Invention

According to the present invention it is also useful to use the pathogenesis preventing carbohydrate together with a glycosidase inhibitor, preferably sialidase inhibitor.

According to the present invention it is also useful to use the pathogenesis preventing carbohydrate together with a lectin or another carbohydrate binding protein. The lectin can be used to block carbohydrate receptors, for example on the bacterial exopolysaccharides.

Glycolipid and carbohydrate nomenclature is according to recommendations by the IUPAC-IUB Commission on Biochemical Nomenclature (Carbohydrate Res. 1998, 312, 167; Carbohydrate Res. 1997, 297, 1; Eur. J. Biochem. 1998, 257, 29).

It is assumed that Gal, Glc, GlcNAc, and Neu5Ac are of the D-configuration, Fuc of the L-configuration, and all the monosaccharide units in the pyranose form. Glucosamine is referred as GlcN or GlcNH₂ and galactosamine as GalN or GalNH₂. Glycosidic linkages are shown partly in shorter and partly in longer nomenclature, the linkages of the Neu5Ac-residues α3 and α6 mean the same as α2-3 and α2-6, respectively, and with other monosaccharide residues α1-3, β1-3, β1-4, and β1-6 can be shortened as α3, β3, β4, and β6, respectively. Lactosamine refers to N-acetyllactosamine, Galβ4GlcNAc, and sialic acid is N-acetylneuraminic acid (Neu5Ac, NeuNAc or NeuAc) or N-glycolylneuraminic acid (Neu5Gc) or any other natural sialic acid. Term glycan means here broadly oligosaccharide or polysaccharide chains present in human or animal glycoconjugates, especially on glycolipids or glycoproteins. In the shorthand nomenclature for fatty acids and bases, the number before the colon refers to the carbon chain lenght and the number after the colon gives the total number of double bonds in the hydrocarbon chain. Abbreviation GSL refers to glycosphingolipid. Abbreviations or short names or symbols of glycosphingolipids are given in the text and in Tables 1 and 2. Helicobacter pylori refers also to the bacteria similar to Helicobacter pylori.

The expression “terminal oligosaccharide sequence” or “terminal sequence” indicates that the oligosaccharide sequences or sialic acid derivative sequences are not linked to other monosaccharide or oligosaccharide structures except optionally from the reducing end of the oligosaccharide sequence. The oligosaccharide sequence when present as conjugate is preferably conjugated from the reducing end of the oligosaccharide sequence, though other linkage positions which are tolerated by the pathogen binding can also be used. In a more specific embodiment the oligosaccharide sequence according to the present invention means the corresponding oligosaccharide residue which is not linked by natural glycosidic linkages to other monosaccharide or oligosaccharide structures. The oligosaccharide residue is preferably a free oligosaccharide or a conjugate or derivative from the reducing end of the oligosaccharide residue.

The term “α3/β3” indicates that the adjacent residues in an oligosaccharide sequence can be either α3- or β3-linked to each other.

The present invention is further illustrated by the following examples, which in no way are intended to limit the scope of the invention:

Experimental Section

Ganglioside Preparations—For isolation of gangliosides a number of tissues, previously described to contain complex gangliosides, as e.g. human erythrocytes, bovine erythrocytes, rabbit thymus, human meconium and human cancers (Stults et al., 1989), were collected. Isolation of total acid glycosphingolipid fractions was done as described previously (Karlsson 1987). Briefly, the tissues were lyophilised, followed by extraction in two steps with chloroform/methanol (2:1 and 9:1, by volume) in a Soxleth apparatus. The material obtained was pooled, subjected to mild alkaline hydrolysis and dialysis, followed by separation on a silicic acid column. Acid and non-acid glycosphingolipids were separated on a DEAE column.

The acid glycosphingolipid fractions were separated by DEAE-Sepharose chromatography, followed by repeated silicic acid chromatography, and final separation was achieved using HPLC on Kromasil 5 silica column of length 250 mm, inner diameter 10 mm and particle size of 5 μm (Phenomenex, Torrance, Calif., USA) using linear gradients of chloroform/methanol/water (60:35:8 to 40:40:12 or 65:25:4 to 40:40:12, by volume) over 180 min, with a flow rate of 2 ml/min. The 2 ml fractions collected were analyzed by thin-layer chromatography and anisaldehyde staining (see below), and the H. pylori binding activity was assessed using the chromatogram binding assay (see below). The fractions were pooled according to the mobility on thin-layer chromatograms and their H. pylori binding activity.

Reference Glycosphingolipids—Reference glycosphingolipids were isolated and characterized at the Institute of Medical Biochemistry, Göteborg University, Sweden. Structural characterisation was performed using proton NMR (Koerner et al., 1983), mass spectrometry (Samuelsson et al., 1990) and degradation studies (Yang and Hakomori 1971, Stellner, 1973). Sialyl-Le^{x} hexaglycosylceramide was purchased from ARC, Edmonton, Canada.

Bacterial Strains, Growth Conditions and Labeling—H. pylori strain CCUG 17874 was obtained from the Culture Collection University of Göteborg (CCUG. Strain J99 was kindly provided by Drs. Tim Cover, John Atherton and Martin Blaser.

Bacteria were grown on Brucella medium (Difco Laboratories, Irvine, Calif.) containing 10% fetal calf serum (Harlan Sera-Lab Loughborough, UK) inactivated at 56° C., and BBL IsoVitale X Enrichment (Becton Dickson Microbiology Systems, Franklin Lakes, N.J.). Bacteria were radiolabelled by the addition of 50 μCi ³⁵S-methionine (Amersham Pharmacia Biotech, Little Chalfont, U.K) diluted in 0.5 ml phosphate-buffered saline (PBS) pH 7.3, to the culture plates. After incubation for 12-72 h at 37° C. under microaerophilic conditions, the bacteria were harvested and centrifuged three times at 3,500 rpm for 10 min in PBS.

Alternatively, colonies were inoculated (1×10⁵ CFU/ml) in Ham's F12 medium (Invitrogen Corp., Carlsbad, Calif., UK), supplemented with 10% heat-inactivated fetal calf serum and 50 μCi ³⁵S-methionine. The culture bottles were incubated with shaking under microaerophilic conditions at 37° C. for 24 h. Bacterial cells were harvested by centrifugation, and washed three times with PBS.

In both cases, the bacteria were finally resuspended in PBS containing 2% (w/v) bovine serum albumin (PBS/BSA) to approximately 1×10⁸ CFU/ml. Both labeling procedures resulted in suspensions with specific activities of approximately 1 cpm per 100 H. pylori organisms.

Thin-Layer Chromatography—Total acid glycosphingolipid fractions (40 μg) or pure gangliosides (0.0002-4 μg) were separated on aluminium backed silica gel 60 HPTLC plates (Merck, Darmstadt, Germany) using chloroform/methanol/water (60:35:8, by volume), chloroform/metanol/0.25% KCl in water (50:40:10, by volume) or 1-propanol/water/25% NH₃ (7:3:1, by volume) as the solvent systems. Chemical detection of glycosphingolipids on thin-layer chromatograms was carried out using anisaldehyde (Waldi 1962) or resorcinol (Svennerholm 1963) reagents.

Chromatogram Binding Assay—The chromatogram binding assay was essentially carried out as described previously (Ångström et al., 1994). Dried thin-layer chromatograms with separated glycosphingolipids were treated in 0.5% polyisobutylmethacrylate (w/v) (Aldrich Chemical Company Inc., Milwaukee, Wis.) in diethylether/n-hexane (1:5, by volume) for 1 min and then air dried. To reduce non-specific binding plates were incubated in PBS/BSA containing 0.1% NaN₃ (w/v) and 0.1% Tween 20 (by volume) at room temperture for 2 h. The plates were then incubated for 2 h at room tempeture with ³⁵S-labeled H. pylori diluted in PBS/BSA. Binding of ¹²⁵]-labeled cholera toxin B-subunits to dilutions of the GM1 ganglioside on thin-layer chromatograms was done as described (Karlsson and Strömberg, 1987). Following the final wash and drying, autoradiography was carried out over night using Biomax film (Eastman Kodak Company, NY, USA). To assess the quantity of bacterial binding to one pure ganglioside relative to others, binding of bacteria to ganglioside dilutions on thin-layer chromatograms and autoradiography was followed by densitometry of the autoradiographs. The range of ganglioside concentrations were in each case chosen on the basis of pilot experiments where the concentrations giving saturation were determined. The autoradiographs were replicated using a CCD camera (Dage-MTI, Inc., Michigan City, Ind.), and analysis of the images was performed using the public domain NIH Image program (developed at the U.S. National Institutes of Health, and available at http://rsb.info.nih.gov/nih-image. Data are presented after subtraction of background values.

Derivatisations of Gangliosides—Gangliosides were permethylated using sodium hydroxide and methyl iodide in dimethyl sulphoxide as described previously described (Larsson et al., 1987). Reduction of permethylated samples was carried out using LiAlH₄ in diethylether (Karlsson 1974).

Fast Atom Bombardment and Electron Ionisation Mass Spectroscopy—Negative ion FAB and EI mass spectra were obtained on a JEOL SX 102A mass spectrometer (JEOL, Toyko, Japan). Negative ion FAB mass spectra of native gangliosides were obtained using Xe atom bombardment (6 eV), an acceleration voltage of −8 kV and triethanolamine as matrix. EI spectra of derivatized glycosphingolipids were obtained with an ionisation voltage of 70 eV, an ionsiation current of 300 μA and an acceleration voltage of 8 kV. The temperature was raised from 150° C. to 410° C. at a rate of 10° C./min. For the collection of both FAB and EI spectra a resolution of 1000 was used.

Electrospray Ionisation Mass Spectrometry and Collision Induced Dissociation—The permethylated H. pylori-binding ganglioside of human erythrocytes was dissolved in 10% acetonitrile in water (by volume). Electrospray ionisation was carried out on a Q-TOF mass spectrometer (Micromass, UK) using the nanoflow electrospray option with a static flow rate of approximately 15-40 nL/min. A spray was generated by the application of 900V across the nanospray needle. Source temperature was 80° C. Nitrogen at a flow rate of 150 l/h was used as drying gas. The doubly charged peak at m/z 1594.4 observed in the mass spectrum was chosen for collision induced dissociation. Collision induced dissociation was achieved using argon with a collision energy of 30-80V. Data were collected over a mass range of 50-2600 mass units. The nomenclature of Domon and Costello (Domon and Costello, 1988) was used in this context.

Proton NMR Spectroscopy—¹H NMR spectra were acquired on Varian 500 MHz and 600 MHz spectrometers at 30° C. The samples were dissolved in dimethyl sulphoxide/D₂O (98:2, by volume) after deuterium exchange.

Special materials and methods for detailed epitope dissection experiments

Source of Natural Glycolipids

PGCs (human erythrocytes) were isolated by us according to the peracetylation method (Miller-Podraza et al. 1993). S-3-PG (human erythocytes and human leukocytes), disialylparagloboside (human erythrocytes), S-6-PG (human leukocytes), 7-sugar neolacto ganglioside (human erythrocytes and leukocytes) and globoside (human erythrocytes) also were prepared in our laboratory (Karlsson 1987). GQ1b of human brain was from Department of Neurochemistry of Göteborg University (Miller-Podraza et al. 1992). Gangliosides GM1, GD1a, GD1b and GT1b of bovine brain were purchased from Calbiochem (USA).

Source of Carbohydrates

NeuAcα3Gαβ4GlcNAcβ3Galβ4Glc was prepared in our laboratory from S-3-PG (human erythrocytes) using ceramide glycanase (from leech, Boehringer Mannheim GmBH, Germany) digestion (Ito and Yamagata 1989) and phase partition in chloroform/methanol/water, 2:1:0.6. The pentasaccharide was recovered from the upper phase. NeuAcα3Galβ3GlcNAcβ3Galβ4Glc, NeuAcα6Galβ4GlcNAcβ3Galβ4Glc and Galβ3(NeuAcα6)GlcNAcβ3Galβ4Glc were from IsoSep (Tullinge, Sweden)

Other Reagents

4-Hexadecylaniline, methylamine, ethylamine, propylamine butylamine and benzylamine were from Aldrich Chemical Company (Milwaukee, USA). Glycolic acid, ethanolamine and octadecylamine were purchased from Sigma-Aldrich (Germany). Sephadex LH 20 was from Pharmacia (Uppsala, Sweden) and ethylene glycol from Fluka (Sweden).

Chemical Modifications of the Sialic Acid Glycerol Tail

A. Mild Periodate Oxidation (Veh et al. 1977) Followed by Reduction (R—CHOH—CHOH—CH₂OH->R—CHOH—CH₂OH/R—CH₂OH):

The material (0.5-1 μmol) was incubated in 500 μl of 0.05 mM acetate buffer, pH 5.5, containing 1-2 mM NaIO₄, for 40 min on ice. The reaction was terminated with an excess of ethylene glycol. The sample was then concentrated by freeze drying (about 5-fold) and reduced with an excess of NaBH₄ at room temperature, overnight. Finally the sample was dialyzed against distilled water for 2 days and freeze dried.

B. Mild Periodate Oxidation of S-3-PG Followed by Coupling with Methylamine or Ethanolamine (R—CHOH—CHOH—CH₂OH->R—CH₂—NH—CH₃/R—CH₂—NH—CH₂—OH).

After oxidation of S-3-PG with mild periodate (see above) and addition of ethylene glycol, the material was dialyzed for 2 days against distilled water and freeze dried. The oxidized S-3-PG was coupled with methylamine or ethylamine under the following conditions: the glycolipid (0.5 mg) was dissolved in 200 μl of M/C 3:1 and mixed with 50 μl of amine, 200 μl of NaBH₃CN in methanol (62 mg/ml) and 200 μl of glycolic acid in water (136 mg/ml). In the case of methylamine, 50 μl of tetrahydrofuran was added to improve solubility. The sample was incubated at 30° C. for 4 h and evaporated under nitrogen. The residue was suspended in C/M/water, 60:30:4.5, and desalted using Sephadex LH-20 column packed in methanol. After application of the sample (about 0.6 ml per 0.5×15 cm column), the column was eluted with methanol and the glycolipid was recovered by collecting sugar-positive fractions (monitored by TLC and anisaldehyde). For final purification of molecular species (see formulae above), the material was separated by preparative TLC using C/M/water, 60:35:8, as developing system. The main band (detected with anisaldehyde after cutting off a strip from the plate) was scraped out and extracted with the same solvent.

Modifications of the Carboxyl Group (Lanne et al. 1995)

S-3-PG (0.5-5 mg) was first converted to the methylester (R—COOH->R—COCH₃) by incubation with methyl iodide (100 μl) in dimethylsulfoxide (DMSO, 0.5 ml), for 1 h at room temperature. The product was purified using Sephadex LH-20, as described above. To prepare the alcohol derivative (R—COCH₃->R—CH₂OH) of S-3-PG, the methylester (0.5 mg) was dissolved in 0.5 ml of methanol, followed by addition of 5 mg of NaBH₄. After 1 h at room temperature the reduced S-3-PG was desalted using Sephadex LH-20 (see above).

For synthesis of the amide and the methyl-, ethyl-, propyl-, benzyl- and stearylamide of S-3-PG, the methyl ester (0.5 mg in 0.5 ml of methanol) was mixed with: 0.2 ml of 30% NH₃ in water, 0.5 ml of 40% methylamine in water, 1 ml of 70% ethylamine in water, 100 μl of propylamine, 100 μl of benzylamine or 200 μl stearylamine in THF (saturated solution), respectively. After incubation at room temperature overnight, the products were evaporated under nitrogen. Benzyl and stearyl amides were further purified by extraction with hexane/acetone 1:1 (by vol.). The samples were washed with excess of the above solvent mixture, centrifuged and the supernatants were discarded.

PGC derivatives were prepared in the same way with the following exceptions. 1, the reduced PGC preparation was separated on DEAE-Sephadex column and only the neutral fraction was further investigated and 2, the oxidation/reduction procedure was performed twice.

Coupling of Hexadecylaniline to Free Saccharides (30)

Saccharide (0.5 mg) was dissolved in 100 μl of methanol and mixed with 100 μl of NaBH₃CN in methanol (62 mg/ml), 100 μl of hexadecylaniline in tetrahydrofuran (40 mg/ml) and 100 μl of 1.8 M glycolic acid in water (136 mg/ml). Additional 100-200 μl of tetahydrofuran were added to improve solubility of precipitating hexadecylaniline. The sample was incubated at 30° C. overnight and the product purified by Sephadex LH chromatography (see “modifications of the carboxyl group” above). The yield of this reaction was more than 90%.

Synthesis of Branched Lipid Parts (Magnusson et al. 1994, Read et al. 1977)

In order to obtain neoglycolipids with branched lipid parts the HDA-derivatized saccharides were further modified by N-acylation. 500 μl of p-nitrophenylpalmitate in dry DMSO was added to 200-300 μg of the dried HDA-saccharide. Four drops of triethylamine were added to the sample which was incubated in nitrogen atmosphere at 37° C. for 3 days. Glycolipids were purified using Sephadex LH-20 column chromatography (see above) and preparative TLC. The yield was about 20%.

Preparation of Lactones

S-3PG was transformed into its lactone form by the method described by Laferriére and Roy (33). 1 mg of S-3PG (1 mg) was dissolved in concentrated acetic acid, and the reaction was allowed to proceed at room temperature for two days. The acetic acid was then evaporated and the remaining material dissolved in C/M/water, 60:35:8 (0.5 ml), by volume. The yield of the reaction was controlled by thin-layer chromatography on aluminium-backed HPTLC plates coated with 0.1 mm silica gel 60 (Merck, Germany) and was about 50%.

Synthesis and Preparation of Ganglioside GM1b

Gangliotetraosylceramide, prepared by desialylation of the ganglioside GM1, was sialylated at the terminal galactose using an α-2,3-sialyltransferase (EC number 2.4.99.4). The conditions were as described before (Lee et al. 1994) with slight modifications, as follows. Gangliotetraosylceramide (100 μg) was dissolved in 10 pd of 500 mM MES (4-morpholineethane-sulfonic acid) buffer, pH 6.0, containing Triton CF-54 (2%) and diluted with 74.1 μl water. Then, 10 μl of CMP-NeuAc (15 mM in water) and 6 μl of α-2,3-sialyltransferase (338 mU/ml) were added to the reaction mixture and the sample was left at room temperature over night. The progress of the reaction was analysed by TLC using C/M/0.25% KCl_{aq} (50:40:10, by volume) as eluent. The reaction mixture was then evaporated under a stream of nitrogen, dissolved in C/M/H₂O, (60:30:4.5, by volume, (2 ml) and applied to a small column (ca 2.5×0.6 cm) packed with Sephadex G-25 (prewashed with 5 ml of the same solvent system). The glycolipid material was eluted with 2.5 ml of the above solvent mixture and 2.5 ml C/M, (2:1, by vol.). Finally, the material was evaporated and dissolved in a small volume of the C/M/H₂O, (60:30:4.5).

Helicobacter pylori Strains

H. pylori strain CCGU 17874 was from Culture Collection Göteborg University, Sweden, and H. pylori strain 032 was a gift from Prof. T. Wadström from Department of Medical Microbiology, Lund University, Sweden. The conditions of bacterial growth in Ham's F12 liquid medium and on Brucella agar plates, respectively, were as described previously (Miller-Podraza et al. 1996). In this paper we used the strain CCGU 17874 from agar plates for expressing the binding to S-3-PG (linear structure) and the strain 032 from liquid medium for expressing binding to PGCs (branched structures).

Results

Gangliosides Preparations—To be able to dissect the ganglioside binding preferences of H. pylori a ganglioside library was compiled (summarized in Table I). Each ganglioside was characterized by mass spectrometry and proton NMR. The procedure is illustrated by the following description of the isolation and characterization of one H. pylori-binding ganglioside of human erythrocytes (No. 19 in Table I).

Total acid glycosphingolipids were isolated from 500 l of pooled blood group B erythrocytes by standard procedures (Karlsson 1987), yielding 5.5 g. A sub-fraction of 390 mg were separated on a 700 ml DEAE-Sepharose column eluted with a linear gradient using 2100 ml of ammonium acetate in methanol (0.05 M to 0.45 M). Each 10 ml fraction collected was analyzed by thin-layer chromatography using the resorcinol reagent. The fractions were pooled according to the mobility of the major compounds. Pooling of fractions 62-67 yielded 15.4 mg, and the fraction obtained had a major compound migrating in the sialyl-neolactotetraosylceramide region. However, when tested for H. pylori-binding activity using the chromatogram binding assay, a slow-migrating binding-active compound was detected. The 15.4 mg of acid glycosphingolipids were further separated by HPLC using a linear gradient of chloroform/methanol/water (60:35:8 to 40:40:12, by volume). The H. pylori-binding compound eluted in fractions 39-56, which after pooling yielded 0.9 mg.

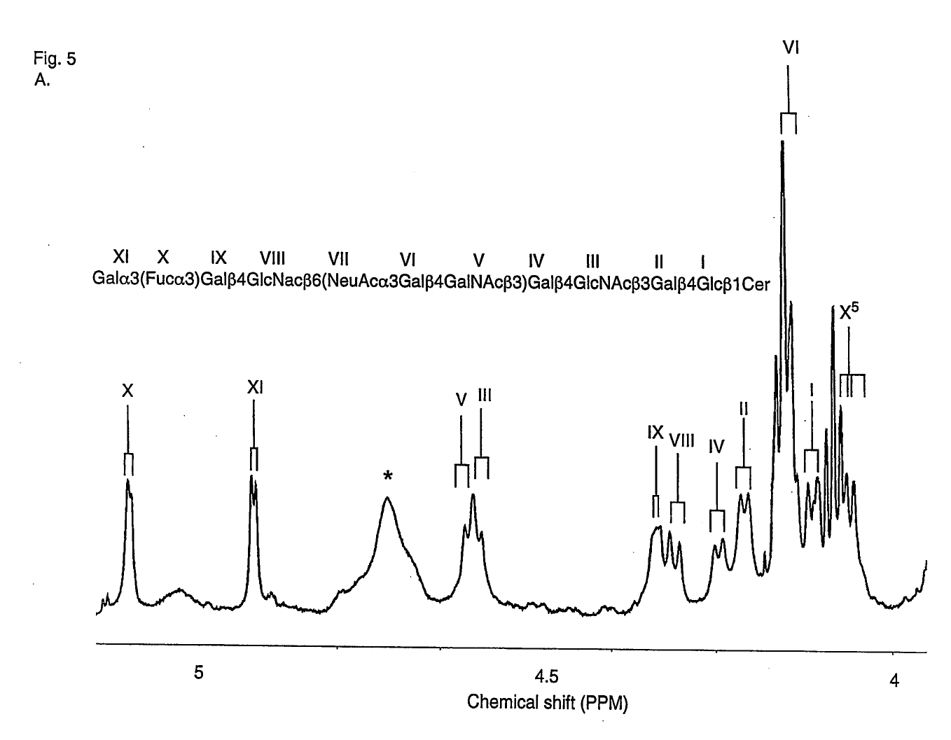

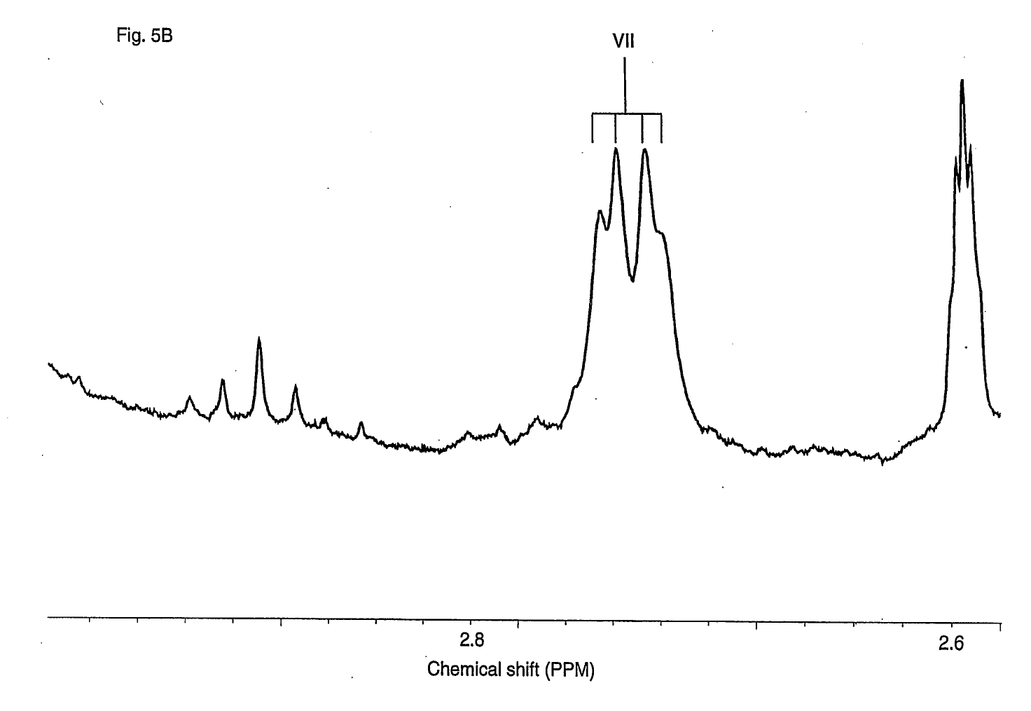

The characterization of the glycolipid structure is shown in FIGS. 1-5 and will be published more in detail elsewhere.

Binding of H. pylori to the Ganglioside Library

The H. pylori strains CCUG 17874 and J99, used in the chromatogram binding experiments, are both sialic acid binding (Mahdavi et al., 2002).

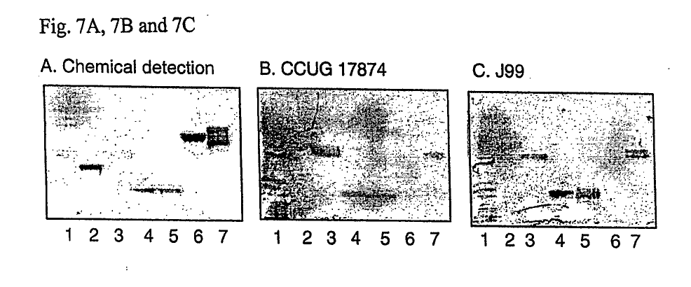

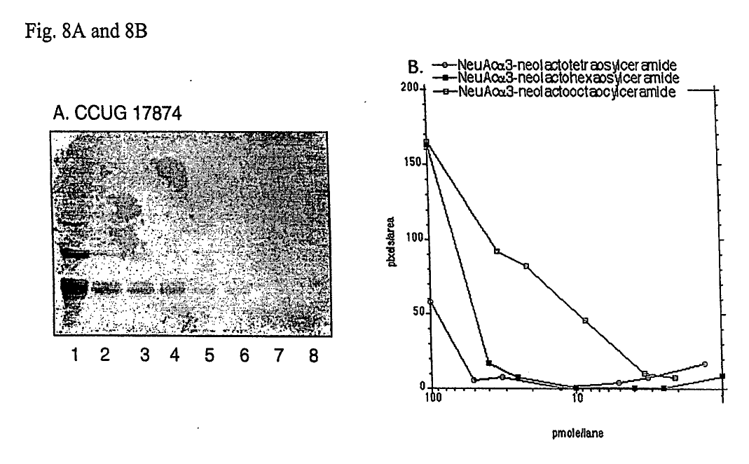

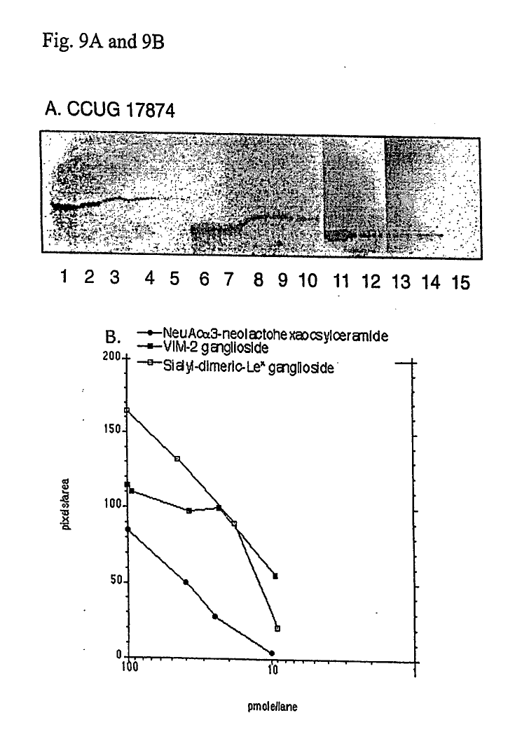

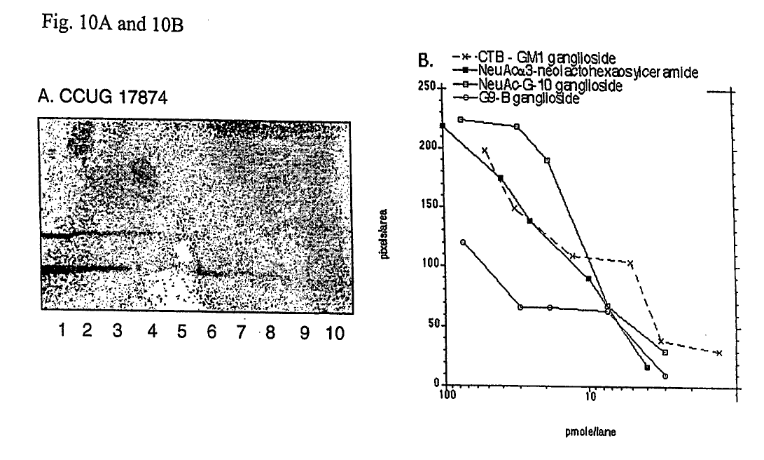

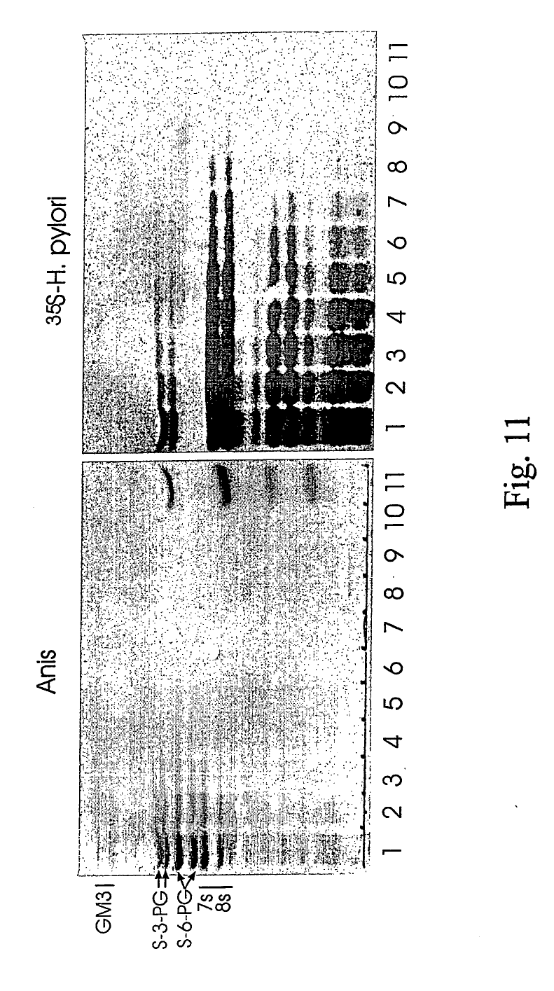

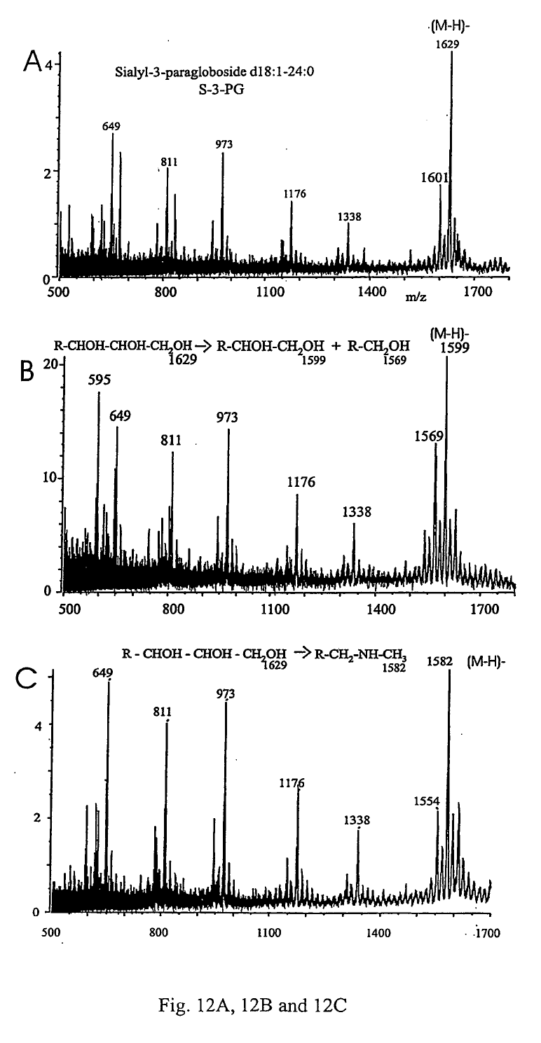

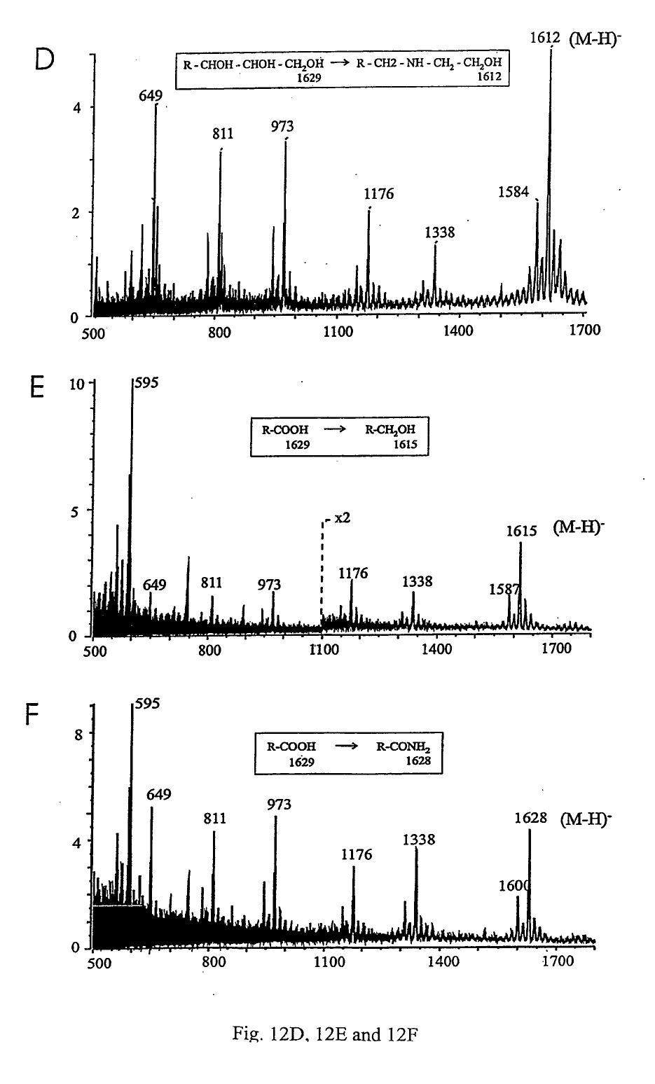

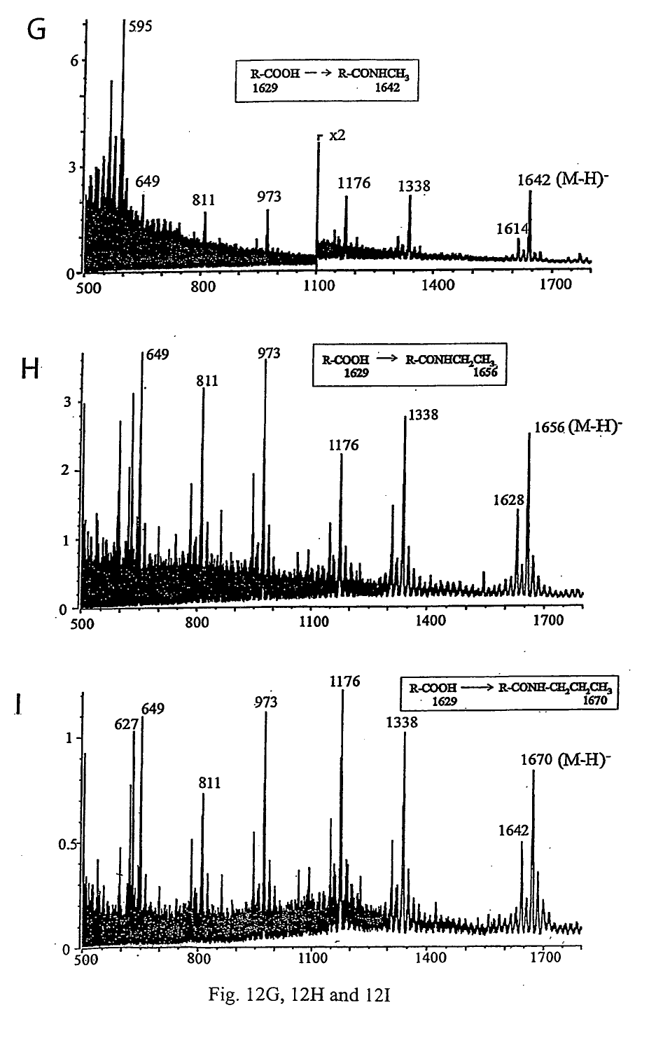

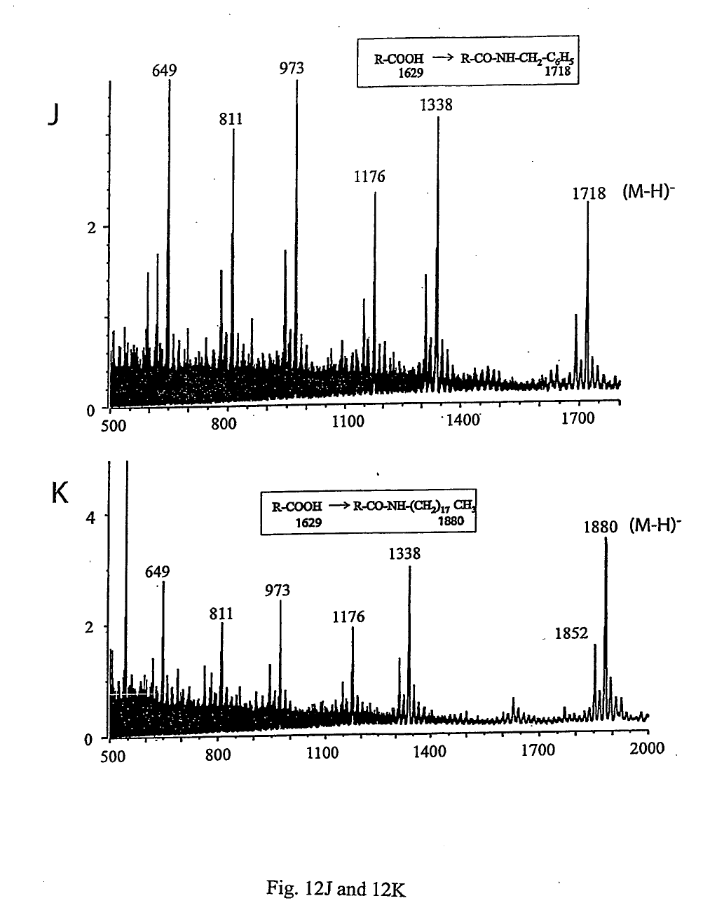

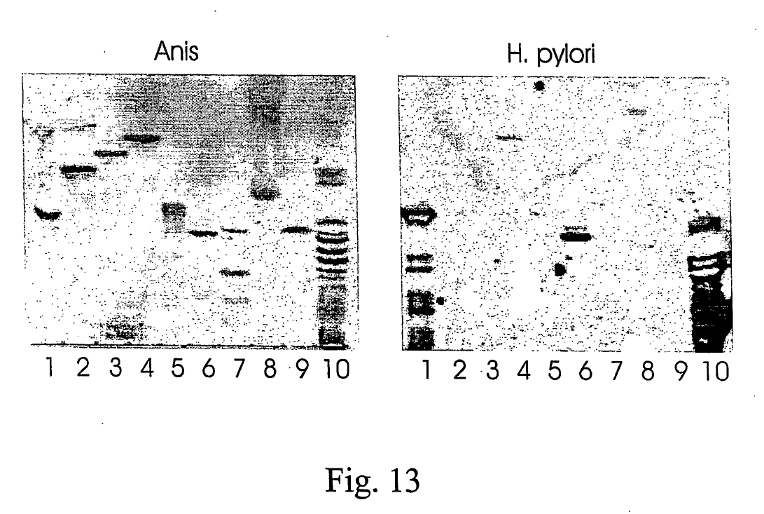

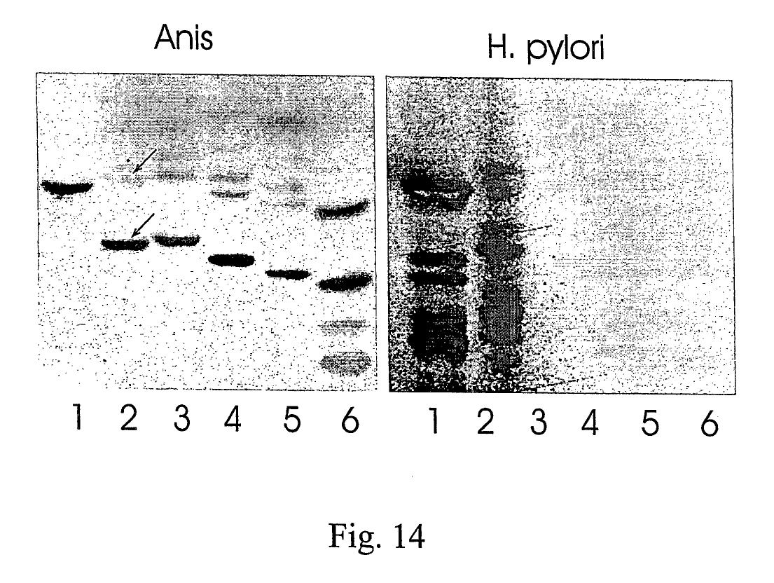

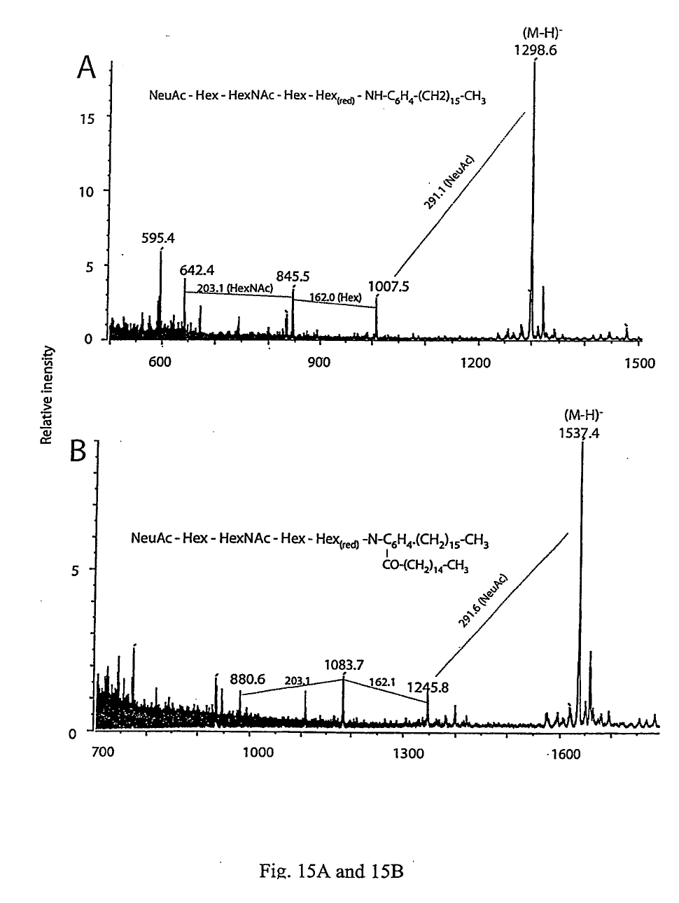

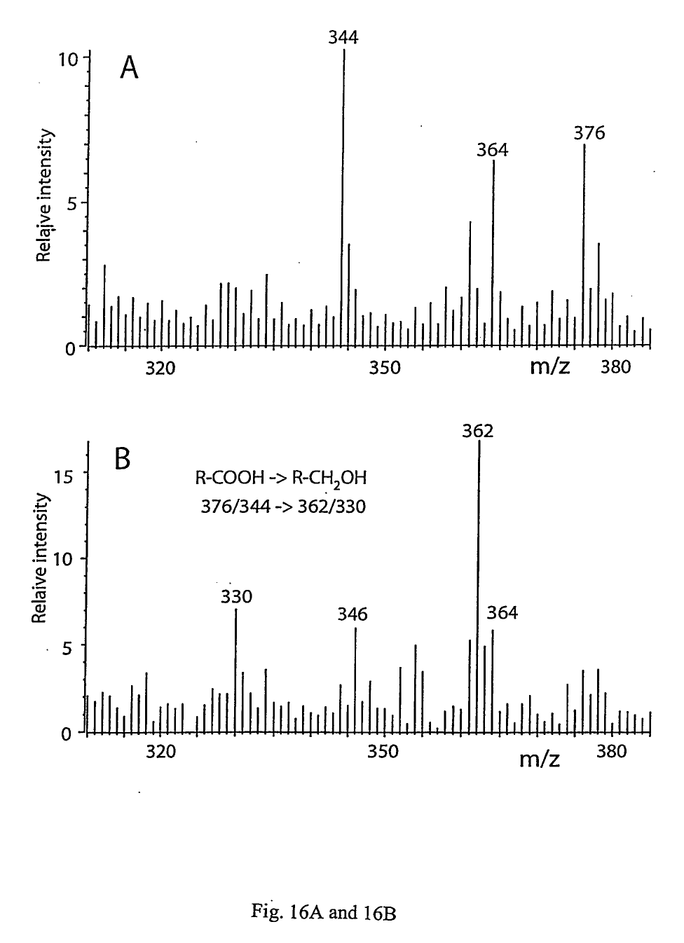

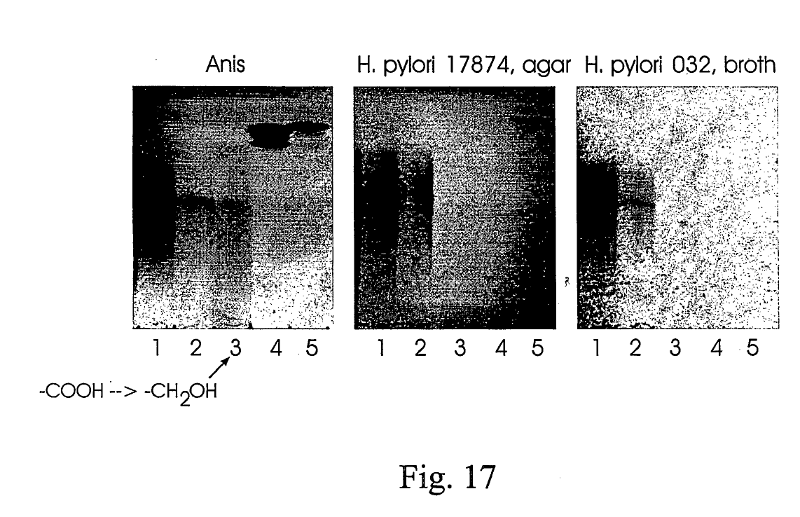

I. Binding and Non-Binding Gangliosides—The results from binding of the H. pylori strains to the isolated gangliosides are exemplified in FIGS. 6-10, and summarized in Table I. Gangliosides were classified as non-binding when no binding was obtained although 4 μg of the compound was applied on the thin-layer plates. As shown in FIG. 6 the sialic acid-binding wild type strains CCUG 17874 and J99 recognized the NeuAc-terminated gangliosides NeuAcα3Galβ4GlcNAcβ3Galβ4GlcNAcβ3Galβ4Glcβ1Cer (NeuAc-neolactohexaocylceramide; lane 1), NeuAcα3Galβ4GlcNAcβ6 (NeuAcα13Galβ4GlcNAcβ3)Galβ4GlcNAcβ3Galβ4Glcβ1Cer (NeuAc-G-10 ganglioside; lane 3), Galα3(Fucα2)Galβ4GlcNAcβ6 (NeuAcα3Galβ4GlcNAcβ3)Galβ4GlcNAcβ3Galβ4Glcβ1Cer (G9-B ganglioside; lane 6), but not the corresponding NeuGc-terminated isostructures (lanes 2, 4 and 5). Further gangliosides recognized were NeuAcα3(Galβ4GlcNAcβ3)₃Galβ4Glcβ1Cer (NeuAc-neolactooctaaocylceramide, FIG. 8, lanes 1-7), NeuAcα13Galβ4GlcNAcβ3Galβ4(Fucα3)GlcNAcβ3Galβ4Glcβ1Cer (VIM-2 ganglioside, FIG. 9, lanes 6-10), and NeuAcα3Galβ4(Fucα3)GlcNAcβ3Galβ4(Fucα3)GlcNAcβ3Galβ4Gclβ1Cer (sialyl-dimeric-Le^{x} ganglioside, FIG. 9, lanes 11-14).

An occasional binding to NeuAcα3-neolactotetraosylceramide (No. 2 in Table I; FIG. 8, lanes 1-7) was also detected, while NeuAcα6-neolactotetraosylceramide (No. 3) was non-binding, in line with previous reports (Miller-Podraza et al, 1997, Johansson and Miller-Podraza 1998). The NeuAcα6-carrying gangliosides Galβ4GlcNAcβ6(NeuAcα6Galβ4GlcNAcβ3)Galβ4Glcβ1Cer (No. 14) and Galβ4GlcNAcβ6(NeuAcα6Galβ4GlcNAcβ3)Galβ4GlcNAcβ3Galβ4Glcβ1Cer (No. 15) were also non-binding. No binding to NeuGcα3-neolactotetraosylceramide (No. 4) or disialyl-neolactotetraosylceramide (No. 5) was obtained. An occasional binding to the sialyl-Le^{x} hexaglycosylceramide (No. 7) was observed. The sialyl-Le^{a} hexaglycosylceramide (No. 6) was not recognized by the CCUG 17874 strain, while the J99 strain occasionally bound to this compound.

II. Comparison of Relative Binding Affinities—Binding of radiolabeled H. pylori to glycosphingolipids diluted in microtiter wells was initially attempted in order to appreciate the relative binding affinities for the various binding-active gangliosides. However, the results thereby obtained were not reproducible. Therefore, binding assays using dilutions of gangliosides on thin-layer plates were utilized. In initial experiments we found that the binding to NeuAcα3-neolactohexaosylceramide and the NeuAc-dimeric-Le^{x} ganglioside was satured at approximately 100 pmole, and therefore lower concentration ranges (1-100 pmole) were utilized in order to obtain binding curves. The results presented in FIGS. 8-10 are representative of a large number of binding assays. Although the level of binding varied somewhat between different batches of radiolabeled bacteria, the same relationships between the binding-active gangliosides were repeatedly obtained. To allow a comparison with a well characterized ganglioside recognition system, the result from binding of ¹²⁵I-labeled cholera toxin B-subunits to dilutions of the GM1 ganglioside on a thin-layer chromatogram is included in FIG. 10.

A. Effect of Carbohydrate Chain Length—Binding of H. pylori strain CCUG 17874 to dilution series of NeuAcα3-neolactotetraosylceramide (No. 2 in Table I), NeuAcα3-neolactohexaosylceramide (No. 8), and NeuAcα3-neolactooctaosylceramide (No. 10), demonstrated a clear preference for NeuAcα3-neolactooctaosylceramide (FIG. 8).

B. Effect of Branching—To evaluate the effect of branching of the carbohydrate chain, the binding of H. pylori strain CCUG 17874 to NeuAcα3-neolactohexaosylceramide (No. 8), the NeuAc-G-10 ganglioside (No. 16) and the G9-B ganglioside (No. 19), Table 1, was compared. As shown in FIG. 9 the blood group B type 2 epitope on the β6-linked branch of the G9-B ganglioside impaired the binding when compared to the linear NeuAcα3-neolactohexaosylceramide. On the other hand, since the NeuAc-G-10 ganglioside was the preferred ligand, it would indicate that the NeuAcα3Galβ4GlcNAc sequence on the β6-linked branch in this case increased the binding affinity.

C. Effect of Fucose Residues—To investigate the effect of fucose branches the relative binding of H. pylori strain CCUG 17874 to NeuAcα3-neolactohexaosylceramide (No. 8), the VIM-2 ganglioside (No. 12) and the sialyl-dimeric-Le^{x} ganglioside (No. 13) was assessed. As shown in FIG. 10 the bacteria bound with higher affinity to the VIM-2 ganglioside and the sialyl-dimeric-Le^{x} ganglioside compared to NeuAcα3-neolactohexaosylceramide.

Detailed Epitope Dissection of the Terminal NeuNAcα3LacNAc-Structures

1. Binding Epitope Associated with Linear Carbohydrate Chains

S-3-PG

S-3-PG, chosen as a model compound for chemical derivatizations, was the simplest ganglioside of human neutrophils which bound H. pylori in our overlay assay (FIG. 11). As shown in the figure, human neutrophils contain a mixture of gangliosides with binding affinity for the bacterium and the strength of binding appears to be higher for more complex, slower migrating species. The minimum amount of S-3PG required for a positive reaction in a typical experiment on TLC plates was 20-30 pmols per 1×7 mm spot, which corresponds to 2.84.2 pmol/mm². For some H. pylori batches the sensitivity of S-3-PG detection was even higher reaching the level of 0.3-0.4 pmol/mm².

Chemically Modified S-3-PG

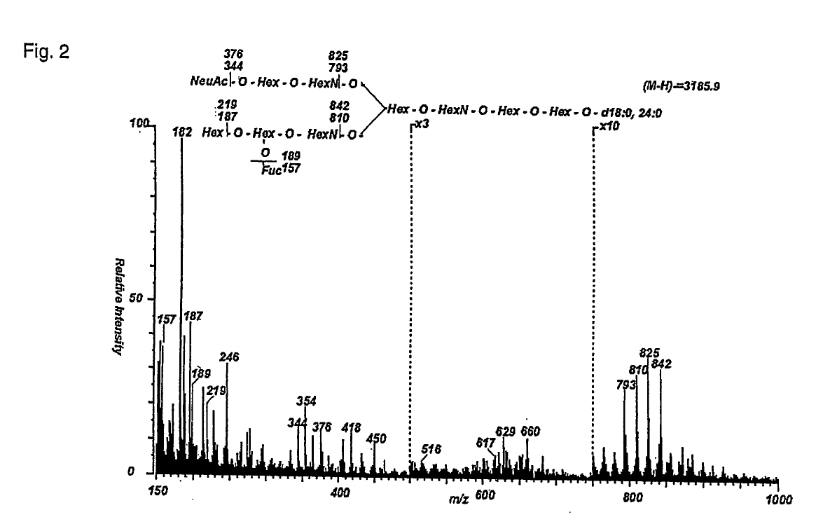

S-3-PG was chemically modified in different ways in order to test the importance of the glycerol tail and the carboxyl group of NeuAc in the interaction with H. pylori. The following derivatizations were performed: (a) mild periodate oxidation of the sialic acid glycerol tail followed by reduction or mild periodate oxidation followed by coupling with methylamine or ethanolamine, (b) reduction of the carboxyl group to primary alcohol, (c) conversion of the carboxyl group to various amides, (d) synthesis of lactones. The derivatives were investigated by negative ion FAB MS in order to confirm the identity of the structures, see FIG. 12. All molecular ions were in agreement with expected masses and the changes were limited to the sialic acid residue (Table 2). The latter was shown by unchanged fragments ions indicating sequence of sugars in the core chain at m/z 1339, 1176, 973 and 811 (Y series of ions according to current nomeclature, Domon and Costello 1988 and Harvey 1999). The undestroyed ceramide part was shown by a fragment ion at m/z 649 (Y₀ ion) (18: 1-24.0). Each of these ions appear together with a satellite ion (−28 mass units) due to the presence of some amounts in the preparation of S-3PG with the d18:1-22:0 ceramide (e.g, m/z 1629 and 1601 in FIG. 2A or 1582 and 1554 in FIG. 2C). The only exception is FIG. 2B where the ions at m/z 1599 and 1569 represent two different derivatives, obtained from the main component 1629 (2A). In FIG. 2B the ions with d:18-22-0 melt together with the background.

All derivatives obtained from S-3PG were tested for binding by H. pylori using overlay of TLC plates with radiolabeled bacteria and the results are summarized in Table 2. An example of binding studies is given in FIG. 13. As shown, most of the modifications performed on the sialic acid residue eliminated or drastically reduced the binding of H. pylori to S-3PG. There was some binding to amide and benzylamide derivatives of S-3PG. Part of the se bindings required higher amounts of the glycolipid material. There was also some interaction with oxidized/reduced SPG on some of the plates (not shown), probably due to the presence of trace amounts non-derivatized SPG in the preparation. To further investigate the importance of the glycerol tail, S-3-PG was oxidized and coupled with methylamine or etanolamine. The two main derivatives obtained were R—CH₂—NH—CH₃ and R—CH₂—NH—CH₂—CH₂OH. Both turned out to be inactive as binding molecules on TLC plates. In contrast, strong binding was observed for octadecylamide of S-3PG which interacted with H. pylori on TLC plates at a lower pmol level (FIG. 14). It was further found out that a C7 derivative of sialic acid oxidized by perjodate from sialic to aldehyde and reductively aminated with octadecylamine binds very effectively to H. pylori. It was further found out that the C1-amide derivatives tolerated the truncation of the glycerol tail of the sialic acid, when the modifications were performed by mild periodate oxidation and reduction of the aldehyde. The data indicates that more active derivatives or analogs can be thus produced or designed from simpler ring formed structures than sialic acid, preferably six atom pyranose like ring structures.

Other Glycolipids and Neoglycolipids

A panel of different natural gangliosides and neogangliosides was tested for interaction with H. pylori (Table 3). There was neither binding of the bacterium to gangliosides of the ganglio series, nor to S-6-PG, NeuAcα8NeuAcα3-PG, or neo-gangliosides prepared from sialylated oligosaccharides based on the lacto (Galβ3GlcNAcβ3Galβ4Glc) core chain. On the other hand, the S-3PG saccharide coupled with HDA or with a branched lipid chain was active under the same overlay conditions. Binding of H. pylori to neoglycolipids synthesized in our lab are shown in FIG. 15. The structures of the main bands were confirmed by mass spectrometry after scraping off the material from the plate. The arrows in lane 2 indicate bands corresponding to the HDA derivative (lower band) and the branched derivative (upper double band). FAB⁻ spectra of these fractions are shown in FIG. 16. The molecular ions and fragment ions were as expected. Similar FAB⁻ spectra were obtained for corresponding fractions shown in lanes 3-5 of FIG. 15. NeuAcα3Galβ4GlcNAcβ3Galβ4Glc saccharide was obtained from a SPG preparation which contained minor amounts of other sialylated molecules of the binding series (lane 1 in FIG. 45, see also FIG. 11). These minor binding fractions are seen even after derivatization (lane 2 in FIG. 15). Repeated experiments showed that reproducibility of binding of H. pylori to HDA neoglycolipids on TLC plates was lower than for derivatives with branched lipid chains.

2. Binding Epitope Associated with Branched Poly-N-Acetyllactosamine Chains of PGCs.

As discussed earlier, this binding could be expressed selectively by some strains of H. pylori grown in liquid cultures. As an example, see FIG. 18 where there is binding to PGCs but not to S-3PG. The minimum amount of PGCs on TLC plates required for a positive reaction in typical experiments was 0.16 pmol NeuAc/mm². The glycerol tail of NeuAc of PGCs was modified using mild periodate oxidation followed by reduction (R—CHOH—CHOH—CH₂OH->R—CHOH—CH₂OH/R—CH₂OH) or coupling with ethanolamine (R—CHOH—CHOH—CH₂OH->R—CH₂—NH—CH₂—OH), and the carboxyl group was modified by reduction (R—COOH->CH₂OH). The modified PGCs were tested by EI MS after permethylation, as exemplified in FIG. 17 which shows ions corresponding to terminal sialic acid residue before (6A) and after reduction (6B). As expected, fragments ions at m/z 376 and 344 representing NeuAc were replaced by ions at m/z 362 and 330, corresponding to reduced NeuAc.

Binding tests using overlay of TLC plates with radiolabeled bacteria showed that the modifications influenced negatively interaction of PGCs with H. pylori. An example of binding of H. pylori to modified PGCs is shown in FIG. 18 and the results are summarized in Table 4.

Discussion

Recognition of sialic acid-containing glycoconjugates by certain H. pylori strains has been repeatedly demonstrated (Roche et al., 2001, Mahdavi, et al., 2002, Miller-Podraza et al, 1997, Johansson and Miller-Podraza 1998). In the present study a library of gangliosides was collected and used for dissection of H. pylori binding preferences utilizing representative sialic acid-recognizing H. pylori.

An occasional binding of the J99 strain to sialyl-Le^{a} hexaglycosylceramide was observed, while the CCUG 17874 strain did not recognize this ganglioside. Binding of the J99 strain to both sialyl-Le^{a}- and sialyl-Le^{x}-neoglycoproteins has also been demonstrated (Mahdavi, 2002). This indicates that the SabA carbohydrate binding sites of J99 strain and the CCUG 17874 strain are not identical. However, in all other respects the CCUG 17874 strain and the J99 strain bound to gangliosides in an identical manner, and both strains recognized N-acetyllactosamine-based gangliosides with terminal NeuAcα3, but not NeuAcα6, in line with previous reports (Miller-Podraza et al, 1997, Johansson and Miller-Podraza 1998).

Furthermore, gangliosides with terminal NeuGcα3 or NeuAcα8NeuAcα3 were not recognized.

Factors that affected the binding affinity were identified as i) length of the N-acetyllactosamine carbohydrate chain, ii) branches of the carbohydrate chain, and iii) fucose substitution of the N-acetyllactosamine core chain.

1. N-acetyllactosamine core length: A preferential binding of H. pylori to NeuAcα3-neolactooctaosylceramide over NeuAcα3-neolactohexaosylceramide and NeuAcα3-neolactotetraosylceramide was observed. This effect is most likely due to an improved accessibility of the carbohydrate head group when presented on a longer core chain.

2. Divalency: A cooperative binding may account for the increased affinity for NeuAc-G-10 ganglioside, having two NeuAcα3Galβ4GlcNAcβ3 branches, relative to the linear NeuAcα3-neolactohexaosylceramide. This is in agreement with the report of Simon et al. (1997) demonstrating that multivalent albumin conjugates of sialyl-lactose (NeuAcα3Galβ4Glc) inhibited the adherence of H. pylori to epithelial monolayers more effectively than monovalent sialyl-lactose. The present invention shows that oligovalent or polyvalent presentation of sialyl-lactosamine allows effective representation this epitope

The lower binding affinity to, the G9-B ganglioside relative to NeuAcα3-neolactohexaosylceramide shows that the blood group B determinant on the β6-linked branch interfered with the binding process. Still there is no absolute hindrance since the detection level for the G9-B ganglioside was approximately 100 pmole. This suggests that the H. pylori binding determinants are mainly exposed on the β3-axis of the Galα3(Fucα2)Galβ4GlcNAcβ6(NeuAcα3Galβ4GlcNAcβ3)Galβ4GlcNAcβ3Galβ4Glcβ1Cer structure.

3. Fucose branches on the N-acetyllactosamine core: The higher binding affinity for the VIM-2 ganglioside relative to NeuAcα3-neolactohexaosylceramide, suggests that the α3-linked Fuc at the innermost GlcNAc contributes to the high affinity binding of sialyl-dimeric-Le^{x}. This fucose residue may either interact with the carbohydrate binding site of the SabA adhesin, or affect the conformation of the ganglioside providing an optimal presentation of the head group. Resolution of this issue must, however, await the expression and crystalization of the SabA adhesin. The expression of the SabA adhesin is, unlike the Le^{b}-binding BabA adhesin, subjected to phase-variation (Mahdavi et al., 2002). Varying number of bacteria expressing the SabA adhesin within the bacterial cell population used in the binding assays may account for the difficulties in determining an absolute affinity of binding for a given ganglioside. However, in repeated binding assays the same relationships between the binding-active gangliosides were observed. A noteworthy observation is, however, that under optimal circumstances the binding of H. pylori to NeuAcα3-neolactohexaosylceramide is comparable to the binding of cholera toxin B-subunits to the GM1 ganglioside. The choleratoxin binding is an example of very strong protein carbohydrate interaction.

The biological significance of these findings requires further studies. The sialic acid content of the primary target tissue of H. pylori, i.e. the human gastric epithelium, is very low (Madrid et al, 1990). However, it was recently demonstrated that an upregulation of the expression of sialic acid-containing glycoconjugates occurs upon gastric inflammation (Mahdavi et al., 2002). Moreover, several of the H. pylori binding gangliosides are also present in human neutrophils (Miller-Podraza et al., 1999, Stroud et al., 1996 a, 1996b), and it was recently demonstrated that the nonopsonic H. pylori-induced activation of human neutrophils occurs by lectinophagocytosis, i.e. recognition of sialylated glycoconjugates on the neutrophil cell surface by a bacterial adhesin leads to phagocytosis and an oxidative burst with the production of reactive oxygen metabolites (Teneberg, et al., 2001). Thus, the sialic acid binding capacity of H. pylori may have a dual role. On the one hand it mediates adhesion of bacteria to the epithelium in the already diseased stomach, and on the other leads to the activation of neutrophils to an oxidative burst with the production of reactive oxygen metabolites and release of biologically active enzymes, giving rise to further tissue damage.

1. Binding of H. pylori to terminal NeuNAcα3LacNAc on carbohydrate chains We chose S-3PG as a model compound for our studies because this glycolipid is relatively easy to prepare and represents a well defined structure. Recognition of sialic acid-containing glycoconjugates by some strains of H. pylori in vitro has been discussed in many papers (Karlsson 1998, Karlsson 2000, Miller-Podraza et al. 1997a, Johansson and Miller-Podraza 1998, Miller-Podraza et al 1996, Miller-Podraza et al. 1997b, Evans et al. 1988, Hirmo et al. 1996, Simon et al. 1997) and our results are in line with these reports, strongly indicating the preference of H. pylori for α3-linked NeuAc and β4-linked Gal. There was apparently a requirement of neolacto structure, since NeuAcα3Galβ3GlcNAc-R and NeuAcα3Galβ3GalNAc-R were inactive. However, GlcNAc itself does not seem to be an absolute requirement of the binding, because sialyllactose, NeuAcα3Galβ4Glc, was shown by others to weakly inhibit the interaction of H. pylori with sialylated structures (Evans et al. 1988, Hirmo et al. 1996, Simon et al. 1997). Besides, α3 Fuc linked to GlcNAc (tested in sialyl-Lewis^{x} oligosaccharide and glycolipid) and other modifications of GlcNAc like de-N-acetylation (Johansson and Miller-Podraza, under preparation) do not abolish the binding. Thus, the present invention shows that modification of position 2 of Glc or GlcNAc is also possible for longer H. pylori binding oligosaccharides without loss of activity.

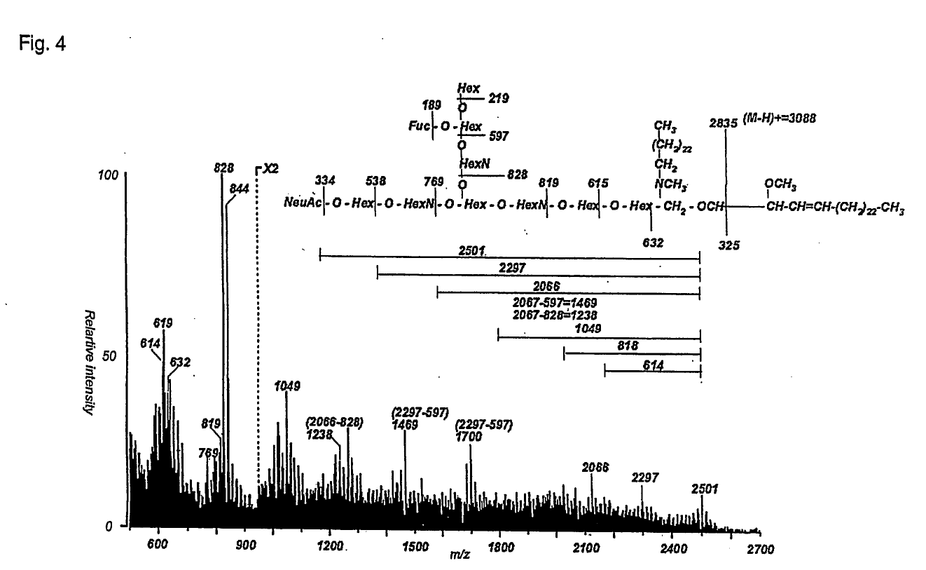

Derivatization of S-3-PG followed by binding studies showed dependence of the binding of H. pylori to sialylated epitopes on the glycerol tail and the carboxyl group. The importance of carboxyl group was demonstrated by reduction of COOH and synthesis of amides. Free amide and benzylamide showed some binding activity for H. pylori on TLC plates, however these reactions were not always reproducible indicating reduced affinity. Large hydrophobic moiety in benzylamide and the positively charged NH₂ group in free amide could provoke unspecific bindings. On the other hand the binding to octadecylamide of SPG was at a lower pmol level (FIG. 4) indicating a specific interaction. The fact that free carboxyl may be replaced by amide form in octadecylamide indicates that only one oxygen of the carboxyl is necessary for the interaction.

We think that NeuAcα3Galβ4GlcNAc, which is part of many human and animal glycoconjugates, represents the optimal terminal part of the natural sialylated binding saccharide for H. pylori. Of importance is that human neutrophils which are actively involved in H. pylori associated infections (Rautelin et al. 1993, Fiocca et al. 1994), are especially enriched in S-3-PG and other neolacto gangliosides (Fukuda et al. 1985, Müthing 1996, Stroud et al. 1996). As mentioned, S-3-PG was the simplest ganglioside from human neutrophils which bound H. pylori on our TLC plates. FIG. 11 shows, that there is a relatively stronger binding to more complex gangliosides. This strong interaction depends most probably on better presentation of the epitopes on TLC surface. During our work we have noticed that the length of the sugar chain as well as the structure of the lipid part may influence the binding. For example, binding of H. pylori to neoglycolipids with hexadecylaniline was less reproducible than binding to neoglycolipids: with branched lipid chains and the binding to sialoneohexaosylceramide was stronger than binding to sialoneotetraosylceramide, as judged from TLC plates (FIG. 11). However other factors should also be taken into account like repeated epitope units, fucose branches or other substitutions. The strong binding to larger species may also depend on combined binding of different bacterial adhesins recognizing both terminal and internal parts the extended core chains. H. pylori is known to display several different binding specificities associated with both sialylated and neutral saccharide chains (Karlsson 1998). Recently Roche N. et al. reported binding of H. pylori to gangliosides with repeated lactosamine units prepared from human gastric carcinoma (Roche et al. 2001).

2. Binding epitope associated with branched poly-N-acetyllactosamine chains We have previously shown that NeuAc in H. pylori-binding PGCs is associated with short branches based on one lactosamine unit (NeuAc-Hex-HexNAc). This was proven by digestion of PGCs by endo-β-galactosidase and analysis of the released oligosaccharide fragments using various mass spectrometry techniques (Karlsson et al. 2000). The presence of NeuAcα3Galβ4GlcNAc in branched PGCs provides favourable conditions for the formation of hydrogen bonds between different sugars and a hypothesis was established that a new binding epitope is created based on interaction between C9 of the glycerol tail of NeuAc and GlcNAcs of the two neighbouring branches (Ångström et al, submitted). Such hypothesis is in agreement with the importance of the glycerol tail for the interaction and can explain the apparent existence of two different binding modes of H. pylori to sialylated structures. The present paper confirms our previous findings on the importance of the glycerol group (Miller-Podraza et al. 1996) and shows that even carboxyl group is crucial for the binding. This is interesting since COOH provides possibilities of rapid in vivo switching off and on of the binding by lactonization or other reversible modifications.

TABLE I
Ganglioside library and results on Helicobacter pylori binding activity

| | | | CCUG | |
|---|---|---|---|---|
| No. | Trivial name | Structure | 17874. | Source |
| 1. | NeuAc-GM3 | NeuAcα3Galβ4Glcβ1Cer | −^{a} | Human |
| | | | | meconium |
| 2. | NeuAcα3SPG | NeuAcα3Galβ4GlcNAcβ3Galβ4Glcβ1Cer | + | Human |
| | | | | erythrocytes |
| 3. | NeuAcα6SPG | NeuAcα6Galβ4GlcNAcβ3Galβ4Glcβ1Cer | − | Human |
| | | | | meconium |
| 4. | NeuGcα3SPG | NeuGcα3Galβ4GlcNAcβ3Galβ4Glcβ1Cer | − | Rabbit |
| | | | | thymus |
| 5. | NeuAc-DPG | NeuAcα8NeuAcα3Galβ4GlcNAcβ3Galβ4Glcβ1Cer | − | Human kidney |
| 6. | NeuAcα3-Le^{a} | NeuAcα3Galβ3(Fucα4)GlcNAcβ3Galβ4Glcβ1Cer | − | Human |
| | | | | gallbladder |
| | | | | cancer |
| 7. | NeuAcα3-Le^{x} | NeuAcα3Galβ4(Fucα3)GlcNAcβ3Galβ4Glcβ1Cer | + | Commercial |
| 8. | NeuAcα3-nLc₆ | NeuAcα3Galβ4GlcNAcβ3Galβ4GlcNAcβ3Galβ4Glcβ1Cer | +++ | Human |
| | | | | hepatoma |
| 9. | NeuGcα3-nLc₆ | NeuGcα3Galβ4GlcNAcβ3Galβ4GlcNAcβ3Galβ4Glcβ1Cer | − | Rabbit |
| | | | | thymus |
| 10. | NeuAcα3-nLc₈ | NeuAcα3Galβ4GlcNAcβ3Galβ4GlcNAcβ3Galβ4GlcNAcβ3Galβ4Glcβ1Cer | +++ | Human |
| | | | | erythrocytes |
| 11. | NeuGcα3-nLc₈ | NeuGcα3Galβ4GlcNAcβ3Galβ4GlcNAcβ3Galβ4GlcNAcβ3Galβ4Glcβ1Cer | − | Rabbit |
| | | | | thymus |
| 12. | VIM-2 | NeuAcα3Galβ4GlcNAcβ3Galβ4(Fucα3)GlcNAcβ3Galβ4Glcβ1Cer | +++ | Human colon |
| | | | | cancer |
| 13. | S-dimer-Le^{x} | NeuAcα3Galβ4(Fucα3)GlcNAcβ3Galβ4(Fucα3)GlcNAcβ3Galβ4Glcβ1Cer | +++ | Human |
| | | | | gallbladder |
| | | | | cancer |
| 14. | | Galβ4GlcNAcβ6(NeuAcα6Galβ4GlcNAcβ3)Galβ4Glcβ1Cer | − | Bovine |
| | | | | buttermilk |
| 15. | | Galβ4GlcNAcβ6(NeuAcα6Galβ4GlcNAcβ3)Galβ4GlcNAcβ3Galβ4Glcβ1Cer | − | Human |
| | | | | meconium |
| 16. | NeuAc-G-10 | NeuAcα3Galβ4GlcNAcβ6(NeuAcα3Galβ4GlcNAcβ3)Galβ4GlcNAcβ3Galβ4Glcβ1Cer | +++ | Human |
| | | | | erythrocytes |
| 17. | NeuGc-G-10 | NeuGcα3Galβ4GlcNAcβ6(NeuGcα3Galβ4GlcNAcβ3)Galβ4GlcNAcβ3Galβ4Glcβ1Cer | − | Bovine |
| | | | | erythrocytes |
| 18. | | Galα3Galβ4GlcNAcβ6(NeuGcα3Galβ4GlcNAcβ3)Galβ4GlcNAcβ3Galβ4Glcβ1Cer | − | Bovine |
| | | | | erythrocytes |
| 19. | G9-B | Galα3(Fucα2)Galβ4GlcNAcβ6(NeuAcα3Galβ4GlcNAcβ3)Galβ4GlcNAcβ3Galβ4Glcβ1Cer | +++ | Human |
| | | | | erythrocytes |

^{a}Binding is defined as follows: +++ denotes a binding when less than 0.5 μg of the glycosphingolipid was applied on the thin-layer chromatogram, while + denotes an occasional binding at 0.5 μg, and − denotes no binding even at 4 μg.

TABLE 2
Binding of Helicobacter pylori to S-3-PG with modified —COOH
and —CHOH—CHOH—CH₂OH.

| | | | Sialic acid fragment | | |
|---|---|---|---|---|---|
| | | M − H | | Calculated | |
| S-3-PG derivative | Chemical modification | Found | Found | (accurate) | Binding |
| Unmodified | | 1629.0 | 291.2 | 291.1 | + |
| Reduced | R—COOH −> R—CH₂OH | 1615.3 | 277.4 | 277.1 | − |
| Amide | R—COOH −> R—CONH₂ | 1628.0 | 290.2 | 290.1 | (+) |
| Methylamide | R—COOH −> R—CONH—CH₃ | 1641.9 | 304.2 | 304.1 | − |
| Ethylamide | R—COOH −> R—CONH—CH₂CH₃ | 1656.3 | 318.5 | 318.1 | − |
| Propylamide | R—COOH −> | 1670.2 | 332.3 | 332.2 | − |
| | −> R—CONH—CH₂CH₂CH₃ | | | | |
| Benzylamide | R—COOH −> R—CONH—CH₂C₆H₅ | 1718.5 | 380.7 | 380.2 | (+) |
| Octadecylamide | R—COOH −> R—CONH—(CH₂)₁₇CH₃ | 1880.1 | 542.3 | 542.6 | + |
| Oxidized/reduced | R—CHOH—CHOH—CH₂OH −> | 1599.2 | 261.3 | 261.1 | − |
| | −> R—CHOH—CH₂OH + | 1569.8 | 231.9 | 231.1 | |
| | + R—CH₂OH | | | | |
| Oxidized/coupled with | R—CHOH—CHOH—CH₂OH −> | 1582.3 | 244.1 | 244.1 | − |
| CH₃NH₂ | −> R—CH₂NH—CH₃ | | | | |
| Oxidized/coupled with | R—CHOH—CHOH—CH₂OH −> | 1612.2 | 274.3 | 274.1 | − |
| OHCH₂CH₂NH₂ | −> R—CH₂NH—CH₂CH₂OH | | | | |
| As lactone | | 1611.2 | 273.3 | 273.1 | − |

TABLE 3
Binding of Helicobacter pylori to various glycolipids on TLC plates. HDA, hexadecylaniline

| | Binding of | |
|---|---|---|
| Glycolipid | H. pylori | Source |
| NeuAcα3Galβ4GlcNAcβ3Galβ4GlcCer, S-3-PG | + | Human erythrocytes |
| NeuAcα3Galβ4(Fucα3)GlcNAcβ3Galβ4GlcCer (Sialyl-Lewis^{x}) | + | Synthesis |
| NeuAcα6Galβ4GlcNAcβ3Galβ4GlcCer, S-6-PG | − | Human leukocytes |
| NeuAcα3Galβ3GlcNAcβ3Galβ4Glc-HDA (or -branched lipid) | − | Chemical synthesis |
| NeuAcα6Galβ4GlcNAcβ3Galβ4Glc-HDA (or -branched lipid) | − | Chemical synthesis |
| Galβ3(NeuAcα6)GlcNAcβ3Galβ4Glc-HDA (or -branched lipid) | − | Chemical synthesis |
| NeuAcα3Galβ4GlcNAcβ3Galβ4Glc-HDA (or -branched lipid) | + | Chemical synthesis |
| NeuAcα3Galβ4GlcNAcβ3Galβ4GlcNAcβ3Galβ4GlcCer | + | Human erythrocytes |
| NeuAcα3Galβ3GalNAcβ4(NeuAcα3)Galβ4GlcCer, GD1a | − | Bovine brain |
| NeuAcα8NeuAcα3Galβ4GlcNAcβ3Galβ4GlcCer | − | Human erythrocytes |
| Galβ3GalNAcβ4(NeuAcα3)Galβ4GlcCer, GM1a | − | Bovine brain |
| NeuAcα3Galβ3GalNAcβ4Galβ4GlcCer, GM1b | − | Enzymatic synthesis |
| Galβ3GalNAcβ4(NeuAcα8NeuAcα3)Galβ4GlcCer, GD1b | − | Bovine brain |
| NeuAcα3Galβ3GalNAcβ4(NeuAcα8NeuAcα3)Galβ4GlcCer | − | Bovine brain |
| NeuAcα8NeuAcα3Galβ3GalNAcβ4(NeuAcα8NeuAcα3)Galβ4GlcCer, GQ1b | − | Human brain |

TABLE 4
Binding of H. pylori (032 strain cultivated in broth) to derivatized
PGCs. R stands for the rest of the PGC molecule.

| PGC Preparation | Chemical modification | Binding |
|---|---|---|
| Unmodified | | + |
| Reduced | R—COOH −> R—CH₂OH | − |
| Oxidized/reduced | R—CHOH—CHOH—CH₂OH −> R—CHOH—CH₂OH + R—CH₂OH | − |
| Oxidized/coupled with | R—CHOH—CHOH—CH₂OH −> R—CH₂—NH—CH₂—CH₂OH | − |
| OHCH₂CH₂NH₂ | | |

### REFERENCES

Borén, T., Falk, P., Roth, K. A. and Normark, S. (1993) Science 262, 1892-1895.
Domon, B. and Costello, C. E. (1988) Glycoconj. J. 5, 397-409
Evans, D. G., Evans, D. J., Jr., Moulds, J. J. and Graham, D. Y. (1988) Infect. Immun. 56(11): 2896-2906.
Fiocca, R., Luinetti, O., Villani, L., Chiaravalli, A. M., Capella, C. and Solcia, E. (1994) Scand. J. Gastroenterol. 205(29S):11-21.
Fukuda, M. N., Dell, A., Oates, J. E., Wu, P., Klock, J. C. and Fukuda, M. (1985) J. Biol. Chem. 260:1067-1082.
Harvey, D. J. (1999) Mass Spectrometry Reviews 18: 349-450.
Hirmo, S., Kelm, S., Schauer, R., Nilsson, B. and Wadström, T. (1996) Glycoconjugate J. 13:1005-1011.
Ilver, D. et al. (1998) Science 279, 373-.
Ito, M. and Yamagata, T. (1989) Methods Enzymol. 179:488-496.
Johansson, L., Miller-Podraza, H. (1998) Anal. Biochem. 265, 260-268.
Karlsson, K.-A. (1987) Meth. Enzymol. 138,212-220
Karlsson, K.-A. (1974) Biochemistry 13, 3643-3647
Karlsson, K.-A. (1998) Mol. Microbiol. 29:1-11.
Karlsson, K.-A. (2000) Glycobiology 10(8): 761-771.
Karlsson, H., Larsson, T., Karlsson, K.-A. and Miller-Podraza, H. (2000) Glycobiology 10(12):1291-1309.
Karlsson, K.-A. and Strömberg, N. (1987) Meth. Enzymol. 138, 220-231
Koerner, T., A., W., Jr., Prestegard, J., H., Demou, P. C. and Yu, R. K. (1983) Biochemistry 22, 2676-2687
Larsson, G., Karlsson, H., Hansson, G. C. and Pimlott, W. (1987) Carbohydr. Res. 161, 281-290
Lanne, B., Uggla, L., Stenhagen, G. and Karlsson, K.-A. (1995) Biochemistry 34:1845-1850.
Lee, Y.-C., Kojima, N., Wada, E., Kurosawa, N., Nakaoka, T., Hamamoto, T. and Tsuji, S. (1994) J. Biol. Chem. 269(13): 10028-10033.
Lingwood, C. A., Huesca, M. and Kuksis, A. (1992) Infect. Immun. 60, 2470-2474
Madrid, J. F., Ballesta, J., Castells, M. T. and Hernandez, F. (1990) Histochemistry 95, 179-187
Magnusson, G., Chernyak, A. Y., Kihlberg, J. and Kononov, L. O. Synthesis of neoglycoconjugates. In: Neoglycoconjugates, Preparation and applications, (Y. C. Lee and Reiko, T. Lee, eds.), Academic Press, Inc., A Division of Harcourt Brace & Company, San Diego 1994, pp. 53-117.
Mahdavi, J., Sondén, B., Forsberg, L., Hurtig, M., Olfat, F. O., Forsberg, L., Roche, N., Ångström, J., Larsson, T., Teneberg, S., Karlsson, K.-A., Altraja, S., Wadström, T., Kersulyte, D., Berg, D. E., Dubois, A., Petterson, C., Magnusson, K.-E,. Norberg, T., Lindh, F., Lundskog, B. B., Arnqvist, A., Hammarström, L., Borén, T. (2002) Science 297, 573-578
Miller-Podraza, H., Månsson, J.-E. and Svennerholm, L. (1992) Biochim. Biophys. Acta 1124:45-51.
Miller-Podraza, H., Andersson, C. and Karlsson, K.-A. (1993) Biochim. Biophys. Acta 1168: 330-339
Miller-Podraza, H., Abul Milh, M., Bergström, J. and Karlsson, K.-A. (1996) Glycoconjugate J. 13: 453-460.
Miller-Podraza, H., Abul Milh, M., Teneberg, S. and Karlsson, K.-A. (1997a) Infect. Immun. 65, 2480-2482
Miller-Podraza, H., Bergström, J., Abul Milh, M. and Karlsson, K.-A. (1997b) Glycoconjugate J 14:467-471.
Miller-Podraza, H., Bergström, J., Teneberg, S., Abul Milh, M., Longard, M., Olsson, B.-M., Uggla, L. and Karlsson, K.-A. (1999) Infect. Immun. 67, 6309-6313
Müthing, J. (1996) Carbohydrate Research. 290:217-224.
Mysore, J. V., Wiggington, T., Simon, P. M., Zopf, D., Heman-Ackah, L. M. and Dubois, A. (1999) Gastroenterology, 117, 1316-1325
Rautelin, H., Blomberg, B., Fredlund, H., Järnerot, G. and Danielsson, D. (1993) Gut 34:599-603.
Read, B., Demel, R. A., Wiegandt, H. and Van Deenen, L. L. M. (1977) Biochim. Biophys. Acta, 470:325-330.
Roche, N., Ångström, J., Larsson, T. and Teneberg, S. (2001) Glycobiology 11, 935-944
Samuelsson, B. E., Pimlott, W. and Karlsson, K-A. (1990) Meth. Enzymol. 193, 623-646
Simon, P. M., Goode, P. L., Mobasseri, A. and Zopf, D. (1997) Infect. Immun. 65, 750-757
Stellner, K., Saito, H. and Hakomori, S.-i. (1973) Arch. Biochem. Biophys. 155, 464-472
Stroud, M. R., Handa, K., Salyan, M. E. K., Ito, K., Levery, S. B., Hakomori, S.-i., Reinhold, B. B. and Reinhold, V. N. (1996a) Biochemistry 35, 758-769
Stroud, M. R., Handa, K., Salyan, M. E. K., Ito, K., Levery, S. B., Hakomori, S.-i., Reinhold, B. B. and Reinhold, V. N. (1996b) Biochemistry 35, 770-778
Stults, C. L. M., Sweeley, C. C. and Macher, B. A. (1989) Meth. Enzymol. 179, 167-214
Svennerholm, L. (1963) J. Neurochem. 10, 613-623
Teneberg, S., Jurstrand, M., Karlsson, K.-A. and Danielsson, D. (2000) Glycobiology 10, 1171-1181
Teneberg, S., Miller-Podraza, H., Lampert, H. C., Evans Jr., D. J., Evans, D. G., Danielsson, D., and Karlsson, K.-A. (1997) J. Biol. Chem. 272, 19067-19071
Teneberg, S., Leonardsson, I., Karlsson, H., Jovall, P.-Å., Ångström, J., Danielsson, D., Näslund, I., Ljungh, Å., Wadström, T., and Karlsson, K.-A. (2002) J. Biol. Chem. 277, 19709-19719
Veh, R. W., Cornfield, A. P., Sander, M. and Schauer R. (1977) Biochim. Biophys. Acta 486:145-160.
Waldi, D. (1962) in Dünnschicht-Chromatographie (Stahl, E., ed.) pp. 496-515. Springer-Verlag, Berlin
Yang, H. and Hakomori, S.-i. (1971) J. Biol. Chem. 246, 1192-1200 Ångström, J., Teneberg, S. and Karlsson, K.-A. (1994) Proc. Natl. Acad. Sci. USA 91, 11859-11863.

1-59. (canceled)

60. A Helicobacter pylori binding substance comprising a hydrophilic oligosaccharide sequence according to Formula 1
R₁Galβ4GlcNAcβ3{(R₂Galβ4GlcNAcβ6)}ₛ₁Gal{β4Glc[NAc]ₛ₃}ₛ₄
wherein R1 and R2 are terminal mono- or oligosaccharides substituents so that at least one of the substituents is NeuNAcα3; s1, s3 and s4 are independently integers 0 or 1 indicating presence or absence of the structure in { } or in [ ];
with the provision that the oligosaccharide sequence is a free oligosaccharide or part thereof or the oligosaccharide sequence is linked to an aglycon comprising less than 23 carbon atoms;
as a non-reducing end terminal sequence, and Helicobacter pylori binding analogs and derivatives thereof, for use as a medicament.

61. The substance according to claim 60, wherein R1 or R2, when not being NeuNAcα3, indicates terminal substituents linked to position 2 and/or 3 of the terminal Gal according to Formula 2
Hex[NAc]ₜ₁α/β3[(DeoxyHexα2)]ₜ₂
wherein Hex is Gal or Glc, integers t1 and t2 are independently 0 or 1 and α/β means that the linkage is either α or β.

62. The substance according to claim 60, wherein said substance is
NeuNAcα3LacNAcβ3(NeuNAcα3LacNAcβ6)LacNAcβ3LacNAc,
NeuNAcα3LacNAcβ3(NeuNAcα3LacNAcβ3LacNAcβ6)LacNAc,
NeuNAcα3LacNAcβ3LacNAcβ3(NeuNAcα3LacNAcβ6)LacNAc,
NeuNAcα3LacNAcβ3(NeuNAcα3LacNAcβ6)LacNAcβ3Lac,
NeuNAcα3LacNAcβ3(NeuNAcα3LacNAcβ6)LacNAcβ3Gal,
NeuNAcα3LacNAcβ3(NeuNAcα3LacNAcβ6)Lac,
NeuNAcα3LacNAcβ3(NeuNAcα3LacNAcβ6)LacNAc,
NeuNAcα3LacNAcβ3(NeuNAcα3LacNAcβ6)Gal,
NeuNAcα3LacNAcβ3(NeuNAcα3LacNAcβ6)LacNAcβ3(NeuNAcα3LacNAcβ6)LacNAc,
NeuNAcα3LacNAcβ3LacNAcβ3LacNAc,
NeuNAcα3LacNAcβ3LacNAcβ3Lac,
NeuNAcα3LacNAcβ3LacNAcβ3Gal,
NeuNAcα3LacNAcβ3LacNAc,
NeuNAcα3LacNAcβ3Lac, or
NeuNAcα3LacNAcβ3Gal

63. The substance according to claim 60, wherein at least one of N-acetylactosamine residues have been replaced by type 2 N-acetyllactosamine analogous structure or structures according to Formula 3
R₁Galβ4Glc[NAc]ᵤ₁β3{(R₂Galβ4Glc[NAc]ᵤ₂β6)}ₛ₁Gal{β4Glc[NAc]ᵤ₃β3Gal}ₛ₂{β4Glc[NAc]ₛ₃}ₛ₄
wherein R1 and R2 are independently nothing or terminal mono- or oligosaccharides substituents with the proviso that at least one of the substituents is NeuNAcα3 or NeuNAcα3Galβ4Glc[NAc]ᵤ₄β3; integers s1, s2, s3 and s4 are independently 0 or 1, indicating the presence or absence of the structures in [ ] or in { }; integers u1, u2, u3, and u4 are independently 0 or 1 indicating the presence of absence of the N-acetyl groups in the non-reducing end terminal or midchain lactosamine residues with the proviso that at least one of the integers present is 0 and
the Glc(NAc)-units may be branched by Fucα3.

64. The substance according to claim 60, wherein the oligosaccharide sequence is linked to an oligovalent or polyvalent carrier by a reduced monosaccharide residue selected from the group consisting of Glc, GlcNAc, and Gal.

65. The substance according to claim 60, wherein said substance is conjugated to a polysaccharide

66. The substance according to claim 60, wherein said substance is an oligomeric molecule containing at least two or three oligosaccharide chains, or said substance consists of a micelle comprising one or more of the substances as defined in claim 1 or said substance is conjugated to a carrier.

67. The substance according to claim 60, wherein position C1 of reducing end terminal Gal, Glc or GlcNAc of said oligosaccharide sequence (OS) is oxygen linked (—O—) to an oligovalent or a polyvalent carrier (Z), via a spacer group (Y) and via a monosaccharide or oligosaccharide residue or derivative (X), forming the following structure
[OS—O—(X)ₙ—Y]ₘ-Z
where integers m, and n have values m≧1, and n is independently 0 or 1; X is lactosyl-, galactosyl-, poly-N-acetyl-lactosaminyl, or part of an O-glycan or an N-glycan oligosaccharide sequence, Y is a spacer group, a terminal conjugate, a ceramide lipid moiety, or a linkage to Z;
or a derivative of the substance of said structure having binding activity to Helicobacter pylori.

68. A pharmaceutical or nutritional composition comprising a substance of claim 60 for the treatment or prophylaxis of any condition due to the presence of Helicobacter pylori.

69. Use of the substance as defined in claim 60, for the diagnosis of a condition due to infection by Helicobacter pylori.

70. A nutritional additive, food-stuff, food preservetive, or beverage containing the composition or substance according to claim 60.

71. A method for the treatment of a condition due to presence of Helicobacter pylori, wherein a pharmaceutically effective amount of the substance as defined in claim 60 is administered to a subject in need of such treatment.

72. The method of treatment according to claims 71, wherein said substance is a nutritional additive or a part of a nutritional composition.

73. The composition or substance according to the claim 60 for binding or inhibition of Helicobacter pylori.

74. Use of the substance as defined in claim 60 for the production of a nutritional additive or composition for the treatment or prophylaxis of any condition due to the presence of Helicobacter pylori.

75. Use of the substance as defined in claim 60 for the identification of bacterial adhesin.

76. Use of the substance as defined in claim 60 for typing Helicobacter pylori.

77. Use of the substance as defined in claim 60 for Helicobacter pylori binding assays.

78. A Helicobacter pylori binding substance comprising a sialic acid derivative as a non-reducing end terminal sequence with binding affinity towards Helicobacter pylori having the structure
NeuNAc(X—R)αxGalβ4GlcNAcβ3Gal(β4Glc)ₚ₁
wherein x is linkage position of the sialic acid derivative and
wherein X is a linking atom or group bound to C1 of sialic acid, R is H or an organic radical comprising more than 3 carbon atoms; X is —NH forming amide structure with the carboxylic acid group of the sialic acid residue; R is H or a C₄-C₃₀ organic radical comprising a ring structure and/or an aliphatic chain; R is a C₆-C₂₄ organic radical or a C6-24 aliphatic alkyl chain; integers p1 is 0 or 1 indicating the presence or absence of the whole structure in ( ).

79. A topical, washing or cosmetic product comprising at least one of the oligosaccharide sequences defined in the claim 60 when the product is selected from the group consisting of: tooth pastes, mouth wash solutions, tablets, cleanser, disinfectant and chewing gums.

80. The method for remodelling natural food material involving the following steps:
1) releasing saccharides from the material chemically or enzymatically,
2) isolating a crude oligosaccharides fraction enriched with desired saccharides which comprises poly-N-acetyllactosamines,
3) releasing the terminal monosaccharides selected from the group consisting of fucose and/or sialic acid, the release may be performed by mild acid treatment and
4) transferring an α3-linked sialic acid to oligosaccharide by a glycosyltransferase or transsialidase enzym.
